(19) United States
(12) Patent Application Publication
(10) Pub. No.: US 2020/0270574 A1
(43) Pub. Date: Aug. 27, 2020

(54) CD1d and TCR-NKT Cells

(71) Applicant: NantCell, Inc., Culver City, CA (US)

(72) Inventors: Patrick Soon-Shiong, Culver City, CA (US); Kayvan Niazi, Culver City, CA (US)

(21) Appl. No.: 16/649,082

(22) PCT Filed: Sep. 28, 2018
(86) PCT No.: PCT/US2018/053506

§ 371 Date: Mar. 19, 2020

Related U.S. Application Data
(60) Provisional application No. 62/585,498, filed on Nov. 13, 2017.
(60) Provisional application No. 62/565,776, filed on Sep. 29, 2017.

(51) Int. Cl. C12N 5/0783; C07K 16/28; C07K 16/30; C07K 16/32; C07K 14/725; C07K 14/705; C12N 9/22

(52) U.S. Cl. CPC C12N 5/0646; C07K 16/2803; C07K 16/3092; C07K 16/32; C07K 14/7051; C12N 2510/00; C12N 9/22; C07K 2317/24; C07K 2319/03; C12N 2800/80; C07K 14/70521

(57) ABSTRACT

Compositions, methods and uses of genetically modified NKT cells to induce an NKT cell immune response against tumor or to change a microenvironment of the tumor by suppressing an activity of myeloid-derived suppressor cells are presented. In some embodiments, naive NKT cells are obtained from a patient having a tumor, and are genetically engineered to include a chimeric protein, a T cell receptor, a hybrid T cell receptor replacing the endogenous T cell receptor, or one of CD40L and Fas-L. The naive or genetically modified NKT cells can be administered to a cancer patient to trigger and/or boost immune response against the tumor.

This application claims priority to our co-pending WIPO patent application having the serial number PCT/US2018/053,506, filed Sep. 28, 2018, US provisional application having the Ser. No. 62/565,776, filed Sep. 29, 2017, and co-pending US provisional application having the Ser. No. 62/585,498, filed Nov. 13, 2017, both of which are incorporated in their entireties herein.

### FIELD OF THE INVENTION

The field of the invention is immunotherapy, especially as it relates to using naive or genetically modified NKT cells specifically targeting cancer cells or suppressing activity of myeloid-derived suppressor cells.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Natural killer T (NKT) cells are considered a subset of T cells expressing T cell receptor α and β chains, yet share properties of natural killer (NK) cells including several molecular markers associated with NK cells (e.g., NK 1.1.). NKT cells recognize various foreign or self-lipid antigens (or some peptide antigens) associated with CD1d in antigen presenting cells. Upon recognition, NKT cells are activated and produce various types of cytokines and chemokines, by which they play a niche role between the innate and adaptive immune response, especially in autoimmune and infectious diseases.

More recently, attention to NKT cells has been drawn with respect to their role in cancer and immune response against cancer cells. For example, some NKT cells can provide a potent antitumor activity by promoting dendritic cells to prime effector cells via release of various cytokines and further so by upregulating costimulatory molecules. Also, some NKT cells can directly kill protumorigenic cells (e.g., protumerigenic macrophage, etc.). Yet, it has been a challenge to use NKT cells in cancer immunotherapy due to the heterogeneity of NKT cells (e.g., type I and type II NKT cells, etc.) and their apparent contradictory roles in promoting and suppressing an immune response against tumors when activated. Further, their relatively small population (e.g., 0.01-2% of human peripheral blood mononuclear cells) provides another obstacle to target the NKT cells for cancer immunotherapy.

Thus, even though some roles in NKT cells in cancer development and/or immune response against tumor are known, NKT cells have not been used effectively in cancer immunotherapy, possibly due to their heterogeneity, low population density, restriction in antigen recognition, dual function in immune response against cancer cells, and lack of specificity to cancer cells. Thus, there remains a need for improved compositions, methods for and uses of naive or genetically modified NKT cells to specifically target cancer cells and/or changing cancer microenvironment to promote effects of cancer immunotherapy.

### SUMMARY OF THE INVENTION

The inventive subject matter is directed to various compositions of, methods for, and use of naive NKT cells or genetically modified NKT cells expressing a chimeric protein or T cell receptor complex to induce NKT cell immune response, and/or to change the microenvironment of the tumor (e.g., by suppressing activity of myeloid-derived suppressor cells). Thus, one aspect of the subject matter includes a genetically engineered NKT cell that comprises a recombinant nucleic acid encoding a chimeric protein or that comprises a recombinant nucleic acid replacing at least one of a portion of T cell receptor alpha locus and a portion of T cell receptor beta locus, and that the recombinant nucleic acid encodes a chimeric protein. The chimeric protein preferably includes 1) an extracellular single-chain variant fragment that specifically binds a tumor (neo)epitope, a tumor associated antigen, or a self-lipid, 2) an intracellular activation domain, and 3) a transmembrane linker coupling the extracellular single-chain variant fragment to the intracellular activation domain.

Preferably, the recombinant nucleic acid comprises a first nucleic acid segment encoding an extracellular single-chain variant fragment that specifically binds the tumor neoepitope, the tumor associated antigen, or the self-lipid, a second nucleic acid segment encoding an intracellular activation domain, and a third nucleic acid segment encoding a linker between the extracellular single-chain variant fragment and the intracellular activation domain. Most typically, the first, second, and third segments are arranged such that the extracellular single-chain variant fragment, the intracellular activation domain, and the linker form a single chimeric polypeptide.

In a preferred embodiment, the extracellular single-chain variant fragment comprises a V_{L} domain and a V_{H} domain of a monoclonal antibody against the tumor neoepitope, the tumor associated antigen, or the self-lipid. In such embodiments, it is contemplated that the extracellular single-chain variant fragment further comprises a spacer between the V_{L} domain and the V_{H} domain. The intracellular activation domain comprises an immunoreceptor tyrosine-based activation motif (ITAM) that triggers ITAM-mediated signaling in the NKT cell. Alternatively, the intracellular activation domain comprises a portion of CD3ζ or a portion of CD28 activation domain. In some embodiments, the linker comprises a CD28 transmembrane domain or a CD3ζ transmembrane domain. Optionally, the genetically engineered NKT cell can further comprise a T cell receptor that specifically binds to CD1d or a Vα24-Jα18 T cell receptor.

In some embodiments, where the recombinant nucleic acid replacing at least one of a portion of T cell receptor alpha locus and a portion of T cell receptor beta locus is included in the genetically engineered NKT cell, it is contemplated that the portion of T cell receptor alpha locus includes a nucleic acid sequence encoding a variable region of extracellular domain of T cell receptor alpha chain. In such embodiment, the variable region of extracellular domain of T cell receptor alpha chain may include Vα24-Jα18 region of the T cell receptor alpha chain. Further, it is also contemplate that the portion of T cell receptor beta locus includes a nucleic acid sequence encoding a variable region of extracellular domain of T cell receptor beta chain and/or the variable region of extracellular domain of T cell receptor alpha chain includes Val 1 region of the T cell receptor beta chain. The recombinant nucleic acid can replace the at least one of the portion of T cell receptor alpha locus and the portion of T cell receptor beta locus by a targeted genome editing nuclease. Preferably, the targeted genome editing nucleases is Cas9 nuclease.

In another aspect of the inventive subject matter, the inventors contemplate a method of inducing an NKT cell immune response in a patient having a tumor. In this method, a genetically engineered NKT cell including a recombinant nucleic acid encoding chimeric protein is provided. The recombinant protein has 1) an extracellular single-chain variant fragment that specifically binds a CD1-lipid antigen complex, a tumor neoepitope, a tumor associated antigen, or a self-lipid, 2) an intracellular activation domain, and 3) a transmembrane linker coupling the extracellular single-chain variant fragment to the intracellular activation domain. The method continues with administering the genetically modified NKT cell to the patient in a dose and a schedule effective to induce an NKT cell immune response against the tumor. Most typically, wherein the administering the genetically modified NKT cell is performed by intravenous injection or intratumoral injection.

In some embodiments, NKT cell immune response against the tumor comprises reducing a size of the tumor or suppressing activity of myeloid-derived suppressor cell.

Preferably, the recombinant nucleic acid in this inventive subject matter comprises 1) a first nucleic acid segment encoding an extracellular single-chain variant fragment that specifically binds the tumor neoepitope, the tumor associated antigen, or the self-lipid, 2) a second nucleic acid segment encoding an intracellular activation domain, and 3) a third nucleic acid segment encoding a linker between the extracellular single-chain variant fragment and the intracellular activation domain. Most typically, the first, second, and third segments are arranged such that the extracellular single-chain variant fragment, the intracellular activation domain, and the linker form a single chimeric polypeptide. Preferably, the tumor epitope is patient-specific and tumor-specific.

In some embodiments, the extracellular single-chain variant fragment comprises a V_{L} domain and a V_{H} domain of a monoclonal antibody against the tumor neoepitope, the tumor associated antigen, or the self-lipid. In such embodiments, it is preferred that the extracellular single-chain variant further comprises a spacer between the V_{L} domain and the V_{H} domain. In other embodiments, the NKT cell further comprises a T cell receptor that specifically binds to CD1d. In still other embodiments, the NKT cell includes a Vα24-Jα18 T cell receptor.

In some embodiments, the intracellular activation domain comprises an immunoreceptor tyrosine-based activation motif (ITAM) that triggers ITAM-mediated signaling in the NKT cell. In other embodiments, the intracellular activation domain comprises a portion of CD3ζ and/or further comprises a portion of CD28 activation domain. In still other embodiments, the linker comprises a CD28 transmembrane domain or a CD3 transmernbrane domain.

In some embodiments, the NKT cell immune response against the tumor comprising reducing a size of the tumor. In other embodiments, the NKT cell immune response against the tumor comprising suppressing activity of myeloid-derived suppressor cells. In such embodiments, it is preferred that the activity of myeloid-derived suppressor cells is suppressed by inducing a cell death of myeloid-derived suppressor cells.

In some embodiments, the recombinant nucleic acid further encodes at least one of CD40L and Fas-L. Alternatively or additionally, the NKT cell may include another recombinant nucleic acid encoding at least one of CD40L and Fas-L.

Additionally, the methods may further comprise a step of providing a condition to the tumor to express a CD1d on a surface of the tumor. In some embodiments, the condition comprises introducing a nucleic acid composition comprising a first nucleic acid segment encoding a CD1d. In such embodiments, the nucleic acid composition further comprises a second nucleic acid segment encoding p99. In other embodiments, the condition comprises a stress condition to the tumor. In still other embodiments, the condition comprises administering an inhibitor of HDAC to increase CD1d expression in the tumor.

Additionally, the method may further comprise a step of obtaining a NKT cell from a bodily fluid of the patient. In such embodiment, the NKT cells are obtained from the bodily fluid using an antibody against Vα-24, and/or using a portion of CD1d, and/or a portion of CD1d coupled with a lipid antigen, and/or a portion of CD1d coupled with a peptide antigen.

In some embodiments, the method may further comprise a step of enriching the NKT cells using a portion of CD1d or an antibody against Vα-24. In other embodiments, the method may further comprise a step of expanding a population of the genetically modified NKT cells ex vivo. In such embodiments, the expanding comprises treating the enriched NKT cells with a cytokine. Preferably, the cytokine is selected from a group consisting of: IL-12, IL-15, IL-18, and IL-21.

In some embodiments, the recombinant nucleic acid replaces at least one of a portion of T cell receptor alpha locus and a portion of T cell receptor beta locus. In such embodiments, the portion of T cell receptor alpha locus includes a nucleic acid sequence encoding a variable region of extracellular domain of T cell receptor alpha chain. Additionally, the variable region of extracellular domain of T cell receptor alpha chain includes Vα24-Jα18 region of the T cell receptor alpha chain. Also, in such embodiments, the portion of T cell receptor beta locus includes a nucleic acid sequence encoding a variable region of extracellular domain of T cell receptor beta chain. It is preferred that the variable region of extracellular domain of T cell receptor alpha chain includes Vall region of the T cell receptor beta chain.

Still another aspect of the inventive subject matter includes a genetically engineered NKT cell including a recombinant nucleic acid encoding a protein complex or a recombinant nucleic acid replacing at least one of a portion of T cell receptor alpha locus and a portion of T cell receptor beta locus, and that encodes the protein complex. The protein complex includes α chain T cell receptor, a β chain T cell receptor, at least a portion of CD3δ, and at least a portion of CD3γ. At least a portion of the α chain T cell receptor and/or the β chain T cell receptor is specific to a patient-specific, tumor-specific neoepitope, or tumor associated antigen, or self-lipid. Preferably, a portion of the protein complex is encoded by a first nucleic acid segment encoding an α chain T cell receptor and a β chain T cell receptor, in which the portions encoding a and β chain receptor are separated by a first self-cleaving 2A peptide sequence. Also, another portion of the protein complex may be encoded by a second nucleic acid segment encoding at least a portion of CD3δ and at least a portion of CD3γ, in which the portions encoding at least portion of CD3δ and the at least portion of CD3γ are separated by a second self-cleaving 2A peptide sequence. In this embodiment, it is also preferred that the first nucleic acid segment and the second nucleic acid segment are separated by a third self-cleaving 2A peptide sequence. It is preferred that the portion of CD3γ and/or CD3δ comprises an immunoreceptor tyrosine-based activation motif (ITAM). Optionally, the genetically engineered NKT cell may further comprises a T cell receptor that specifically binds to CD1d.

In some embodiments, where the recombinant nucleic acid replacing at least one of a portion of T cell receptor alpha locus and a portion of T cell receptor beta locus is included in the genetically engineered NKT cell, it is contemplated that the portion of T cell receptor alpha locus includes a nucleic acid sequence encoding a variable region of extracellular domain of T cell receptor alpha chain. The portion of T cell receptor alpha locus includes a nucleic acid sequence encoding a variable region of extracellular domain of T cell receptor alpha chain, and/or the variable region of extracellular domain of T cell receptor alpha chain includes Vα24-Jα18 region of the T cell receptor alpha chain. Additionally, the portion of T cell receptor beta locus may include a nucleic acid sequence encoding a variable region of extracellular domain of T cell receptor beta chain. Further, the variable region of extracellular domain of T cell receptor alpha chain includes Vall region of the T cell receptor beta chain.

Preferably, the recombinant nucleic acid replaces the at least one of the portion of T cell receptor alpha locus and the portion of T cell receptor beta locus by a targeted genome editing nuclease. It is contemplated that the targeted genome editing nucleases is Cas9 nuclease.

In still another aspect of the inventive subject matter includes a genetically engineered NKT cell including a first recombinant nucleic acid sequence replacing a portion of T cell receptor alpha locus and encoding a first variable domain and a second recombinant nucleic acid sequence replacing a portion of T cell receptor beta locus and encoding a second variable domain. Most preferably, the first and second domains collectively form a binding motif specific to a patient-specific, tumor-specific neoepitope or a tumor associated antigen.

In some embodiments, wherein the portion of T cell receptor alpha locus includes a nucleic acid sequence encoding a variable region of extracellular domain of T cell receptor alpha chain, In such embodiments, it is also contemplated that the variable region of extracellular domain of T cell receptor alpha chain includes Vα24-Jα18 region of the T cell receptor alpha chain. In other embodiments, the portion of T cell receptor beta locus includes a nucleic acid sequence encoding a variable region of extracellular domain of T cell receptor beta chain. In such embodiments, the variable region of extracellular domain of T cell receptor alpha chain includes Vall region of the T cell receptor beta chain.

In some embodiments, the recombinant nucleic acid replaces the at least one of the portion of T cell receptor alpha locus and the portion of T cell receptor beta locus by a targeted genome editing nuclease. Preferably, the targeted genome editing nucleases is Cas9 nuclease.

In still another aspect of the inventive subject matter, the inventors contemplate a method of inducing an NKT cell immune response in a patient having a tumor. In this method, a genetically engineered NKT cell expressing a protein complex is provided. The protein complex includes at least an α chain T cell receptor, a β chain T cell receptor, at least a portion of CD3δ, and at least a portion of CD3γ. The method further continues with administering the genetically engineered NK cell to the patient in a dose and a schedule effective to induce an NKT cell immune response against the tumor. Most typically, the administering the genetically modified NKT cell is performed by intravenous injection or intratumoral injection. In some embodiments, the NKT cell immune response against the tumor comprises reducing a size of the tumor. In other embodiments, the NKT cell immune response against the tumor comprising suppressing activity of myeloid-derived suppressor cells.

In some embodiments, the first nucleic acid segment and the second nucleic acid segment are separated by a third nucleic acid segment encoding a self-cleaving 2A peptide. In other embodiments, the portion of CD3γ comprises an immunoreceptor tyrosine-based activation motif (ITAM) and/or the portion of CD3δ comprises an immunoreceptor tyrosine-based activation motif (ITAM). In other embodiments, the genetically engineered NKT cells may comprise a T cell receptor that specifically binds to CD1d.

In some embodiments, the method may further comprise a step of co-administering cytokine-induced killer cells with the genetically engineered NKT cells. In other embodiments, the method may further comprise a step of providing a condition to the tumor to express a CD1d on a surface of the tumor. In such embodiments, the condition may comprise introducing a nucleic acid composition comprising a first nucleic acid segment encoding a CD1d, and/or the nucleic acid composition further comprising a second nucleic acid segment encoding p99. Alternatively and/or additionally, the condition may comprise a stress condition to the tumor, and/or administering an inhibitor of HDAC to increase CD1d expression in the tumor.

In some embodiments, the recombinant nucleic acid replaces at least one of a portion of T cell receptor alpha locus and a portion of T cell receptor beta locus. In such embodiments, the portion of T cell receptor alpha locus includes a nucleic acid sequence encoding a variable region of extracellular domain of T cell receptor alpha chain. Preferably, the variable region of extracellular domain of T cell receptor alpha chain includes Vα24-Jα18 region of the T cell receptor alpha chain. Also, in such embodiments, the portion of T cell receptor beta locus includes a nucleic acid sequence encoding a variable region of extracellular domain of T cell receptor beta chain. Preferably, the variable region of extracellular domain of T cell receptor alpha chain includes Val 1 region of the T cell receptor beta chain.

Preferably, the recombinant nucleic acid replaces the at least one of the portion of T cell receptor alpha locus and the portion of T cell receptor beta locus by a targeted genome editing nuclease. In such scenario, it is preferred that the targeted genome editing nucleases is Cas9 nuclease.

In some embodiments, the recombinant nucleic acid encodes at least one of CD40L and Fas-L, and/or the NKT cells further include another recombinant nucleic acid encoding at least one of CD40L and Fas-L.

In still another aspect of the inventive subject matter, the inventors contemplate a method of suppressing an activity of myeloid-derived suppressor cells in a patient having a tumor. In this method, NKT cells are genetically modified to express at least one of CD40L and Fas-L, preferably on their cell surfaces. Then, a plurality of genetically modified NKT cells is administered to the patient in a dose and a schedule effective to suppress the activity of myeloid-derived suppressor cells. Typically, the administering the genetically modified NKT cell is performed by intravenous injection or intratumoral injection.

In some embodiments, the plurality of genetically modified NKT cells is CD1d-restricted T cells. In some embodiments, the genetically modified NKT cells include a recombinant nucleic acid encoding at least one of CD40L and Fas-L.

In some embodiments, the method further comprises a step of providing a condition to the tumor to express a CD1d on a surface of the tumor. The condition may comprise introducing a nucleic acid composition comprising a first nucleic acid segment encoding a CD1d. Preferably, the nucleic acid composition further comprising a second nucleic acid segment encoding p99. The condition may comprise a stress condition to the tumor and/or administering an inhibitor of HDAC to increase CD1d expression in the tumor.

In still another aspect of the inventive subject matter, the inventors contemplate a method of inducing an NKT cell immune response in a patient having a tumor. In this method, a plurality of NKT cells of a patient is obtained from the patient's bodily fluid. Most typically, the bodily fluid is blood. Then, the NKT cells are enriched using a binding molecule specific to the plurality of NKT cells. Preferably, the binding molecule includes an antibody against Vα-24, a portion of CD1d, or a portion of CD1d coupled with a lipid antigen. The population of enriched NKT cells is expanded ex vivo, preferably in the presence of one or more cytokines. Then, the expanded NKT cells are administered to the patient in a dose and a schedule effective to induce an NKT cell immune response against the tumor. Typically, the administering the genetically modified NKT cell is performed by intravenous injection or intratumoral injection. In some embodiments, the NKT cell immune response against the tumor comprises reducing a size of the tumor, and/or suppressing activity of myeloid-derived suppressor cells.

In some embodiments, the method can further include providing a condition to the tumor to express a CD1d on a surface of the tumor. The condition may include introducing first nucleic acid segment encoding a CD1d, preferably with a second nucleic acid segment encoding p99. The condition may also include administering an inhibitor of HDAC to the patient to increase CD1d expression in the tumor.

In some embodiments, the method may further include a step of further enriching the NKT cells using a portion of CD1d, and/or an antibody against Vα-24.

In some embodiments, the step of expanding comprises treating the enriched NKT cells with a cytokine. In such embodiments, the cytokine is selected from a group consisting of: IL-12, IL-15, IL-18, and IL-21.

Still another aspect of the inventive subject matter includes a pharmaceutical composition for treating a patient having a tumor, wherein the pharmaceutical composition comprises a plurality of genetically engineered NKT cells as described above.

Still another aspect of the inventive subject matter includes use of genetically engineered NKT cells as described above or the pharmaceutical compositions described above for treating a tumor of a patient having the tumor.

Various objects, features, aspects and advantages of the inventive subject matter will become more apparent from the following detailed description of preferred embodiments, along with the accompanied drawings.

### BRIEF DESCRIPTION OF THE DRAWING

FIG. 1 shows graphs of flowcytometry data that NKT cells (CD3+Vα24+) are specifically isolated using Vα24 antibody and expanded with treatment of α-galactosylceramide (α-GalCer).

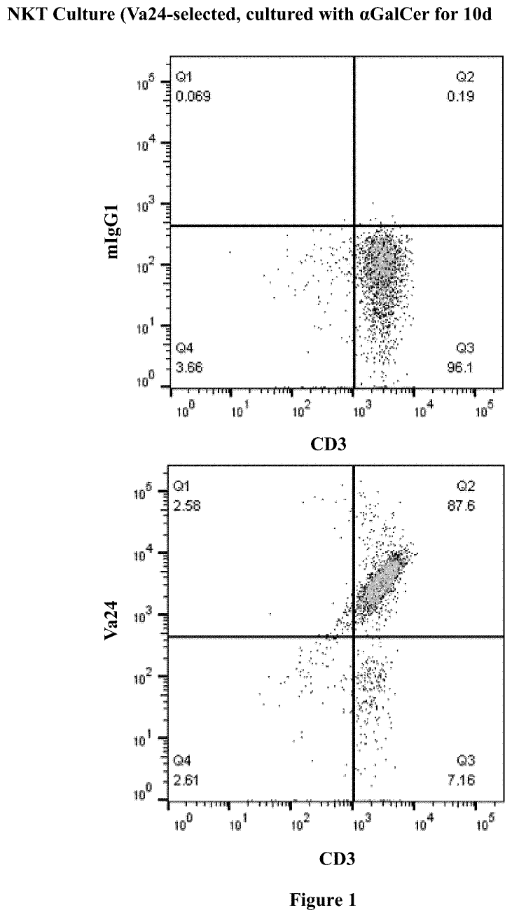

FIG. 2 shows graphs indicating inhibited T cell proliferation by myeloid-derived suppressor cells (MDSCs).

### DETAILED DESCRIPTION

The inventors now discovered that NKT cell immune response can be effectively and specifically induced against a tumor by modifying NKT cells to specifically recognize a tumor specific or tumor associated antigen, a neoepitope, and/or a self-lipid of the tumor, and/or by changing the tumor microenvironment to be more susceptible to an immune response against the tumor.

In order to achieve such goal, the inventors have now discovered that a (homogenous) NKT cell population from a patient can be obtained, isolated, and expanded. The so expanded NKT cells can then be reintroduced to the patient to trigger an immune response of NKT cells against the tumor cells or tumor cell environment. Additionally, the inventors discovered that NKT cells can also be genetically modified to express a receptor that specifically binds to a tumor specific or tumor associated antigen, a neoepitope, and/or a self-lipid of the tumor, which consequently triggers an NKT immune response against the tumor.

As used herein, the term “tumor” refers to, and is interchangeably used with one or more cancer cells, cancer tissues, malignant tumor cells, or malignant tumor tissue, that can be placed or found in one or more anatomical locations in a human body. As used herein, the term “bind” refers to, and can be interchangeably used with a term “recognize” and/or “detect”, an interaction between two molecules with a high affinity with a K_{D} of equal or less than 10⁻⁶M, or equal or less than 10⁻⁷M. As used herein, the term “provide” or “providing” refers to and includes any acts of manufacturing, generating, placing, enabling to use, or making ready to use.

### Isolation NKT Cells

NKT cells represent a heterogeneous cell population that can be grouped into three categories based on presence of several molecular markers (e.g., Vα24, etc.) and/or their reactivity to a ligand (e.g., CD1d-restricted, reactivity to α-galactosylceramide (α-GalCer), etc.). However, the heterogeneity and low fraction of NKT cells (e.g., 0.01-2% of human peripheral blood mononuclear cells) in vivo presents obstacles in utilizing NKT cells in eliciting localized immune response against a tumor in a patient. Thus, in one preferred aspect of the inventive subject matter, the inventors contemplate that NKT cells of a patient can be obtained, isolated and then expanded to be reintroduced to the patient. NKT cells can be obtained from any suitable tissues of a patient, so long as NKT cells are present in the tissue. Most typically, suitable tissue sources include whole blood. It is contemplated that the number of NKT cells expected to be present in the tissue may vary among individuals (e.g., based on age, gender, health status, ethnicity, etc.) and the type of tissue (e.g., whole blood, cerebrospinal fluid, etc.). For example, in order to obtain NKT cells from a patient having a tumor, at least 2 ml, preferably at least 5 ml, more preferably at least 10 ml of whole blood can be obtained from the patient.

While adult's peripheral blood is the most accessible source to obtain NKT cells, the fraction of the NKT cells is generally low, and even lower in the cancer patient's peripheral blood. Thus, additionally and alternatively, it is also contemplated that the patient's NKT can be obtained from the patient's stored umbilical cord blood, where NKT cells are present in a higher concentration than adult's peripheral blood. While it may vary depending on the storage conditions, in order to obtain NKT cells from a patient's umbilical cord blood, at least 0.5 ml, preferably at least 1 ml, more preferably at least 2 ml of umbilical cord blood can be obtained from the patient.

From the bodily fluid of the patient, which includes many different types of cells (e.g., erythrocytes, platelets, neutrophils, lymphocytes, etc.), NKT cells can be isolated using several known molecular markers or their binding molecules. In one embodiment, isolation of human type I NKT cells, which typically express Vα24-Jα18 type T cell receptor, can be performed using an antibody against Vα24 or an antibody against Vα24-Jα18. In other embodiments, isolation of human type I and type II NKT cells, which are typically CD1d-restricted cells, can be performed using a portion of CD1d molecule (preferably the portion that are responsible for a high affinity to NKT T cell receptor), a portion of CD1d molecule coupled with a lipid antigen (e.g., any lipid antigens that are generated from a foreign organism, nutritional substances, or self-lipids generated from the patient that can bind to CD1d, etc.), or a portion of CD1d molecule coupled with a peptide (e.g., p99, etc.). Any suitable pull-down techniques to isolate cells are contemplated. For example, an antibody against Vα24 or an antibody against Vα24-Jα18 (or CD1d molecule with or without lipid or peptide antigen) can be immobilized on a bead (e.g., agarose beads, biotin-coated beads, etc.), and then contacted with the patient's bodily fluid. The inventors contemplate that the majority of cells bound to the bead via antibodies (or CD1d molecule with or without lipid or peptide antigen) would be type I NKT cells. Thus, using this process, NKT cells, preferably, a specific type of NKT cells (e.g., type I NKT cells), can be isolated among other blood cells and among other NKT cells.

In some embodiments, the inventors contemplate that the enriching process can be performed with two or more binding molecules to increase specificity, preferably in two separate and sequential contacting processes. For example, the bodily fluid can be contacted first with beads coated with the antibody against Vα24 or the antibody against Vα24-Jα18. The cells bound to the antibody against Vα24 or the antibody against Vα24-Jα18 can be eluted and then contacted second with beads coated with CD1d molecule (with or without lipid or peptide antigen). For other example, the bodily fluid can be contacted first with beads coated with the CD1d molecule (with or without lipid or peptide antigen). Then the cells bound to the CD1d molecule can be further contacted with beads coated with the antibody against Vα24 or the antibody against Vα24-Jα18.

In a preferred embodiment, the NKT cells bound to the antibodies (or CD1d molecule with or without lipid or peptide antigen) can be eluted in a smaller volume of liquid (e.g., cell culture media, etc.) than the original sample volume (e.g., blood volume, etc.) so that the NKT cells can be enriched after the isolation (e.g., NKT cells in 10 ml volume of blood can be isolated in 0.5 ml volume of cell culture media, resulting in about 20 times enrichment of NKT cells after isolation, etc.). In some embodiments, where NKT cells are isolated via two or more contacting processes, the NKT cells can be further enriched by further reducing the volume of elution media (e.g., cell culture media, etc.) in the second contacting process (e.g., 10 ml original sample volume can be reduced to 2 ml eluted cells in the first contacting process, and then further reduced to 0.5 ml eluted cells in the second contacting process, etc.). It is especially preferred that the NKT cells are enriched at least 5 times, preferably at least 10 times, more preferably at least 20 times compared to the number of cells/volume of original bodily fluid sample.

In other embodiments, the NKT cells, preferably a specific type of NKT cells (e.g., type I NKT cells) can be isolated from other cells in the patient's bodily fluid using flow cytometry (e.g., fluorescence activated cell sorting (FACS), etc.) or magnetic activated cell sorting (MACS). For example, type I NKT cells can be isolated from other cells using fluorescence tagged α-CD3 antibody and α-Vα24 antibody (e.g., fluorescein isothiocyanate (FITC)-α-CD3 antibody and Cy3-α-Vα24 antibody, etc.), or magnetic-particle tagged α-CD3 antibody and α-Vα24 antibody. The inventors also contemplate that the isolated CD3+Vα24+ cells by flow cytometry or MACS can be further enriched using a pull-down assay with beads coated with CD1d molecule (with or without lipid or peptide antigen).

### Genetically Modified NKT Cells

NKT Cell Expressing a Recombinant Chimeric Antigenic Receptor (CAR):

The inventors contemplate that isolated (and/or further expanded) NKT cells can be genetically modified for specific targeting tumor cells and/or increasing the effect of NKT cell immune in suppressing the activity of myeloid-derived suppressor cells. In one aspect of the inventive subject matter, the inventors contemplate that NKT cells can be genetically modified to specifically recognize a tumor specific or tumor associated antigen, a neoepitope, and/or a self-lipid expressed by the tumor cell by introducing a recombinant protein to the NKT cells.

Generally, the recombinant protein is a CAR and includes an extracellular single-chain variant fragment, an intracellular activation domain, and a transmembrane linker coupling the extracellular single-chain variant fragment to the intracellular activation domain. Preferably, the recombinant protein is generated from a single chimeric polypeptide translated from a single recombinant nucleic acid. However, it is also contemplated that that the recombinant protein comprises at least two domains that are separately translated from two distinct recombinant nucleic acid such that at least a portion of the recombinant protein can be reversibly coupled with the rest of the recombination protein via a protein-protein interaction motif.

Thus, in a preferred embodiment, in which the recombinant protein is encoded by a single recombinant nucleic acid, the recombinant nucleic acid includes at least three nucleic acid segments: a first nucleic acid segment (a sequence element) encoding an extracellular single-chain variant fragment that specifically binds to a neoepitope, tumor associated antigen, or self-lipid presented on the tumor cell surface; a second nucleic acid segment encoding an intracellular activation domain; and a third nucleic acid segment encoding a linker between the extracellular single-chain variant fragment and the intracellular activation domain.

In this embodiment, the first nucleic acid segment encoding an extracellular single-chain variant fragment includes a nucleic acid sequence encoding a heavy (V_{H}) and light chain (V_{L}) of an immunoglobulin. In a preferred embodiment, the nucleic acid sequence encoding variable regions of the heavy chain (V_{H}) and the nucleic acid sequence encoding variable regions of the light chain (V_{L}) are separated by a linker sequence encoding a short spacer peptide fragment (e.g., at least 10 amino acid, at least 20 amino acid, at least 30 amino acid, etc.). Most typically, the extracellular single-chain variant fragment encoded by the first nucleic acid segment includes one or more nucleic acid sequences that determine the binding affinity and/or specificity to the tumor neoepitope, tumor associated antigen, or self-lipid. Thus, the nucleic acid sequence of V_{H} and V_{L} can vary depending on sequence of the tumor epitope the recombinant protein may target to.

Any suitable methods to identify the nucleic acid sequence of V_{H} and V_{L} specific to the tumor neoepitope, tumor associated antigen, or self-lipid are contemplated. For example, a nucleic acid sequence of V_{H} and V_{L} can be identified from a monoclonal antibody sequence database with known specificity and binding affinity to the tumor epitope. Alternatively, the nucleic acid sequence of V_{H} and V_{L} can be identified via an in silico analysis of candidate sequences (e.g., via IgBLAST sequence analysis tool, etc.). In some embodiments, the nucleic acid sequence of V_{H} and V_{L} can be identified via a mass screening of peptides having various affinities to the tumor neoepitope, tumor associated antigen, or self-lipid via any suitable in vitro assays (e.g., flow cytometry, SPR assay, a kinetic exclusion assay, etc.). While it may vary depending on the characteristics of tumor epitope, it is preferred that the optimal nucleic acid sequence of V_{H} and V_{L} encodes an extracellular single-chain variant fragment having an affinity to the tumor epitope at least with a K_{D} of at least equal or less than 10⁻⁶M, preferably at least equal or less than 10⁻⁷M, more preferably at least equal or less than 10⁻⁸M. Alternatively, synthetic binders to the tumor epitope may also be obtained by phage panning or RNA display.

While it is preferred that that the first nucleic acid segment includes nucleic acid sequences encoding one of each heavy (V_{H}) and light chains (V_{L}), it is also contemplated that in some embodiments, the first nucleic acid segment includes nucleic acid sequence encoding a plurality of heavy (V_{H}) and light chains (V_{L}) (e.g., two heavy (V_{H}) and light chains (V_{L}) for generating a divalent (or even a multivalent) single-chain variable fragments (e.g., tandem single-chain variable fragments). In this embodiment, the sequence encoding one of each heavy (V_{H}) and light chains (V_{L}) can be linearly duplicated (e.g., V_{H}-linker 1-V_{L}-linker 2-V_{H}-linker 3-V_{L}). It is contemplated that the length of the linkers 1, 2, 3 can be substantially similar or same. However, it is also contemplated that the length of linker 2 is substantially different (e.g., longer or shorter) than the length of linker 1 and/or linker 3.

Alternatively, the inventors also contemplate that the extracellular single-chain variant fragment (V_{H} and/or V_{L} chains) can be substituted with an extracellular domain of T-cell receptor. For example, in some embodiments, the extracellular single-chain variant fragment can be substituted with a portion of a chain, β chain, γ chain, or δ chain of T cell receptor. In other embodiments, the extracellular single-chain variant fragment can be substituted with a combination of at least two of a portion of a chain, β chain, γ chain, or δ chain (e.g., hybrid of a chain and (3 chain, a hybrid of γ chain and δ chain, etc.) of T cell receptor. In this embodiment, the nucleic acid sequence of extracellular domain(s) of T-cell receptor, especially hypervariable region(s) of α, β, γ and/or δ chain can be selected based on the measured, estimated, or expected affinity to the tumor neoepitope, tumor associated antigen, or self-lipid. It is especially preferred that the affinity of extracellular domain of T-cell receptor to the tumor epitope is at least with a K_{D} of at least equal or less than 10⁻⁶M, preferably at least equal or less than 10⁻⁷M, more preferably at least equal or less than 10⁻⁸M.

The recombinant nucleic acid also includes a second nucleic acid segment (a sequence element) encoding an intracellular activation domain of the recombinant protein. Most typically, the intracellular activation domain includes one or more ITAM activation motifs (immunoreceptor tyrosine-based activation motif, YxxL/I-X₆₋₈-YXXL/I), which triggers signaling cascades in the cells expressing these motifs. Any suitable nucleic acid sequences including one or more ITAM activation motifs are contemplated. For example, the sequence of the activation domain can be derived from a NK receptor including one or more ITAM activation motif (e.g., intracellular tail domain of killer activation receptors (KARs), NKp30, NKp44, and NKp46, etc.). In another example, the sequence of the activation domain can be derived from a tail portion of a NKT T-cell antigen receptor (e.g., CD3ζ, CD28, etc.). In some embodiments, the nucleic acid sequence of the intracellular activation domain can be modified to add/remove one or more ITAM activation motif to modulate the cytotoxicity of the cells expressing the recombinant protein.

The first and second nucleic acid segments are typically connected via a third nucleic acid segment encoding a linker portion of the recombinant protein. Preferably, the linker portion of the recombinant protein includes at least one transmembrane domain. Additionally, the inventors contemplate that the linker portion of the recombinant protein further includes a short peptide fragment (e.g., spacer with a size of between 1-5 amino acids, or between 3-10 amino acids, or between 8-20 amino acids, or between 10-22 amino acids) between the transmembrane domain and the extracellular single-chain variant fragment, and/or another short peptide fragment between the transmembrane domain and the intracellular activation domain. In some embodiments, the nucleic acid sequence of transmembrane domain and/or one or two short peptide fragment(s) can be derived from the same or different molecule from which the sequence of intracellular activation domain is obtained.

For example, where the intracellular activation domain is a portion of CD3ζ, the entire third nucleic acid segment (encoding both transmembrane domain and short peptide fragment) can be derived from CD3ζ (same molecule) or CD28 (different molecule). In other embodiments, the third nucleic acid segment is a hybrid sequence, in which at least a portion of the segment is derived from a different molecule than the rest of the segment. In a further example, where the intracellular activation domain is a portion of CD3ζ, the sequence of the transmembrane domain can be derived from CD3ζ and a short fragment connecting the transmembrane domain, and the extracellular single-chain variant fragment may be derived from CD28 or CD8.

In other contemplated embodiments, the recombinant nucleic acid includes a nucleic acid segment encoding a signaling peptide that directs the recombinant protein to the cell surface. Any suitable and/or known signaling peptides are contemplated (e.g., leucine rich motif, etc.). Preferably, the nucleic acid segment encoding an extracellular single-chain variant fragment is located in the upstream of the first nucleic acid segment encoding an extracellular single-chain variant fragment such that the signal sequence can be located in N-terminus of the recombinant protein. However, it is also contemplated that the signaling peptide can be located in the C-terminus of the recombinant protein, or in the middle of the recombinant protein.

Additionally, the recombinant nucleic acid may include a sequence element that controls expression of the recombinant protein, and all manners of control are deemed suitable for use herein. For example, where the recombinant nucleic acid is an RNA, expression control may be exerted by suitable translation initiation sites (e.g., suitable cap structure, initiation factor binding sites, internal ribosome entry sites, etc.) and a poly-A tail (e.g., where length controls stability and/or turnover), while recombinant DNA expression may be controlled via a constitutively active promoter, a tissue specific promoter, or an inducible promoter.

NKT Cell Expressing a Recombinant T Cell Receptor Complex:

Additionally or alternatively, the inventors contemplate that NKT cells can also be genetically modified by introducing a recombinant nucleic acid composition encoding a protein complex to the NKT cells. Most typically, the protein complex includes at least one or more distinct peptides having an extracellular domain of a T cell receptor, and at least one or more distinct peptide of the intracellular domain of T cell co-receptor. For example, one preferred protein complex includes an α chain of a T cell receptor, a β chain of a T cell receptor, at least a portion of CD3δ (preferably cytoplasmic domain), and at least a portion of CD3γ (preferably cytoplasmic domain). In another example, the protein complex may include a γ chain T cell receptor and a δ chain T cell receptor instead of the α and β chains of T cell receptors. Additionally, the protein complex may include one or more ζ-chain substituting for the portion of CD3δ or the portion of CD3γ.

While any suitable forms of recombinant nucleic acid composition to encode the protein complex can be used, the inventors contemplate that the protein complex can be encoded by a single nucleic acid comprising a plurality of segments, each of which encodes a distinct peptide. Thus, in one preferred embodiment, the nucleic acid composition includes a first nucleic acid segment encoding two distinct peptides: an α chain T cell receptor and a β chain T cell receptor (or alternatively, γ chain T cell receptor and δ chain T cell receptor), and a second nucleic acid segment encoding two peptides: at least a portion of one type of T-cell co-receptor (e.g., CD3δ) and at least a portion of another type of T-cell co-receptor (e.g., CD3γ), or alternatively, encoding one or more ζ-chain substituting for the portion of CD3δ or the portion of CD3γ. It is contemplated that each distinct peptide encoded by the first and second nucleic acid segments is a full length protein (e.g., full length alpha and β chain T cell receptor and co-receptors). Yet, it is also contemplated that at least one or more distinct peptides encoded by the first and second nucleic acid segments can be a truncated or a portion of the full length proteins.

Preferably, the first and second nucleic acid segments are mRNAs, each of which comprises two sub-segments of mRNA, which encode T cell receptor (e.g., sub-segment A is an mRNA of a chain T cell receptor and sub-segment B is an mRNA of β chain T cell receptor, etc.), followed by poly A tail. It is further preferred that the two sub-segments of mRNA are separated by nucleic acid sequences encoding a type of 2A self-cleaving peptide (2A). As used herein, 2A self-cleaving peptide (2A) refers any peptide sequences that can provide a translational effect known as “stop-go” or “stop-carry” such that two sub-segments in the same mRNA fragments can be translated into two separate and distinct peptides. Any suitable types of 2A peptide sequences are contemplated, including porcine teschovirus-1 2A (P2A), thosea asigna virus 2A (T2A), equine rhinitis A virus 2A (E2A), foot and mouth disease virus 2A (F2A), cytoplasmic polyhedrosis virus (BmCPV 2A), and flacherie virus (BmIFV 2A). In some embodiments, same type of 2A sequence can be used between two sub-segments of both first and second nucleic acid segments (e.g., first nucleic acid segment: mRNA of α chain receptor-T2A-mRNA of β chain receptor; second nucleic acid segment: mRNA of α chain receptor-T2A-mRNA of β chain receptor). In other embodiments, different types of 2A sequence can be used between two sub-segments of both first and second nucleic acid segments (e.g., first nucleic acid segment: mRNA of α chain receptor-T2A-mRNA of β chain receptor; second nucleic acid segment: mRNA of α chain receptor-P2A-mRNA of β chain receptor).

Additionally, the inventors contemplate that the first and second nucleic acid segments can also be present in a single nucleic acid (mRNA), for example, connected by a 2A sequence. In this embodiment, the sub-segments of first and second nucleic acid segments can be arranged in any suitable order (e.g., a chain-(3 chain-CD3γ-CD3δ, β chain-CD3γ-α chain-CD3δ, etc.), with any suitable combination of same of different 2A sequences (e.g., α chain-T2A-β chain-P2A-CD3γ-F2A-CD3δ, β chain-P2A-CD3γ-T2A-α chain-F2A-CD3δ, etc.), followed by poly A tail at the 3′ of the single mRNA.

With respect to the mRNA sequence of first and second nucleic acid segments, it is preferred that the mRNA sequences are selected based on the sequence of the tumor neoepitope, tumor associated antigen, or self-lipid that the protein complex target to. For example, it is preferred that the peptide encoded by the first nucleic acid segment has an actual or predicted affinity to the tumor epitope at least with a K_{D} of at least equal or less than 10⁻⁶M, preferably at least equal or less than 10⁻⁷M, more preferably at least equal or less than 10⁻⁸M. Any suitable methods to identify the first nucleic acid segment sequence that has high binding affinity to the tumor epitope are contemplated. For example, a nucleic acid sequence of first nucleic acid segment can be identified via a mass screening of peptides having various affinities to the tumor epitope via any suitable in vitro assays (e.g., flow cytometry, SPR assay, a kinetic exclusion assay, etc.).

With respect to recognized antigens it should be noted that all antigens that bind to CD1d are deemed suitable for use herein. Consequently, contemplated antigens especially include one or more tumor associated antigens, self-lipids, and especially tumor neoepitopes. Typically, the tumor associated antigens and neoepitopes (which are typically patient-specific and tumor-specific) can be identified from the omics data obtained from the cancer tissue of the patient or normal tissue (of the patient or a healthy individual), respectively. Omics data typically includes information related to genomics, transcriptomics, and/or proteomics. As used herein, the cancer cells or normal cells (or tissues) may include cells from a single or multiple different tissues or anatomical regions, cells from a single or multiple different hosts, as well as any permutation of combinations.

Omics data of cancer and/or normal cells preferably comprise a genomic data set that includes genomic sequence information. Most typically, the genomic sequence information comprises DNA sequence information that is obtained from the patient (e.g., via tumor biopsy), most preferably from the cancer tissue (diseased tissue) and matched healthy tissue of the patient or a healthy individual. For example, the DNA sequence information can be obtained from a pancreatic cancer cell in the patient's pancreas (and/or nearby areas for metastasized cells), and a normal pancreatic cells (non-cancerous cells) of the patient or a normal pancreatic cells from a healthy individual other than the patient.

In one especially preferred aspect of the inventive subject matter, DNA analysis is performed by whole genome sequencing and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination. Therefore, data sets may include unprocessed or processed data sets, and exemplary data sets include those having BAM format, SAM format, FASTQ format, or FASTA format. However, it is especially preferred that the data sets are provided in BAM format or as BAMBAM diff objects (see e.g., US2012/0059670A1 and US2012/0066001A1). Moreover, it should be noted that the data sets are reflective of a tumor and a matched normal sample of the same patient to so obtain patient and tumor specific information. Thus, genetic germ line alterations not giving rise to the tumor (e.g., silent mutation, SNP, etc.) can be excluded. Of course, it should be recognized that the tumor sample may be from an initial tumor, from the tumor upon start of treatment, from a recurrent tumor or metastatic site, etc. In most cases, the matched normal sample of the patient may be blood, or non-diseased tissue from the same tissue type as the tumor.

Likewise, computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers. Such analysis advantageously reduces false positive neoepitopes and significantly reduces demands on memory and computational resources.

The so obtained neoepitopes may then be subject to further detailed analysis and filtering using predefined structural and expression parameters, and sub-cellular location parameters. For example, it should be appreciated that neoepitope sequences are only retained provided they will meet a predefined expression threshold (e.g., at least 20%, 30%, 40%, 50%, or higher expression as compared to normal) and are identified as having a membrane associated location (e.g., are located at the outside of a cell membrane of a cell). Further contemplated analyses will include structural calculations that delineate whether or not a neoepitope or a tumor associated antigen, or a self-lipid is likely to be solvent exposed, presents a structurally stable epitope, etc.

Consequently, it should be recognized that epitopes can be identified in an exclusively in silico environment that ultimately predicts potential epitopes that are unique to the patient and tumor type. So identified epitope sequences are then synthesized in vitro to generate the corresponding peptides. Thus, it is conceptually possible to assemble an entire rational-designed collection of (neo)epitopes of a specific patient with a specific cancer, which can then be further tested in vitro to find or generate high-affinity antibodies. In one aspect of the inventive subject matter, one or more of the peptide (neo)epitopes (e.g., 9-mers) can be immobilized on a solid carrier (e.g., magnetic or color coded bead) and used as a bait to bind surface presented antibody fragments or antibodies. Most typically, such surface presented antibody fragments or antibodies are associated with a M13 phage (e.g., protein III, VIII, etc.) and numerous libraries for antibody fragments are known in the art and suitable in conjunction with the teachings presented herein. Where desired, smaller libraries may also be used and be subjected to affinity maturation to improve binding affinity and/or kinetic using methods well known in the art (see e.g., Briefings in functional genomics and proteomics. Vol 1. No 2.189-203. July 2002). In addition, it should be noted that while antibody libraries are generally preferred, other scaffolds are also deemed suitable and include beta barrels, ribosome display, cell surface display, etc. (see e.g., Protein Sci. 2006 January; 15(1): 14-27.) In addition, as already discussed above, it should be appreciated that not only patient and tumor specific neoepitopes are deemed suitable, but also all known tumor associated antigens (e.g., CEACAM, MUC-1, HER2, etc.) as well as various self-lipids.

NKT Cell Expressing a Hybrid T Cell Receptor Complex:

Additionally or alternatively, the inventors contemplate that NKT cells can also be genetically modified by introducing recombinant nucleic acid sequences encoding two distinct and separate peptides to form a hybrid T cell receptor complex. Preferably, the peptides replace the Vα24-Jα18 region of the endogenous T cell alpha chain (in chromosome 14 in human being) and the Vβ11 region of the endogenous T cell beta chain (in chromosome 7 in human being). More preferably, the peptides replace at least two of CDR1α-CDR3a (e.g., CDR1α-CDR3α, CDR2 α-CDR3α, CDR1α-CDR2α, etc.) and/or at least two of CDR1β-CDR3β (e.g., CDR1β-CDR3β, CDR2 β-CDR3β, CDR1β-CDR2β, etc.) of endogenous T cell alpha chain and T cell beta chain. Thus, the hybrid T cell receptor complex formed with two distinct, separate peptide is likely lose its specificity (or restriction) to CD1d and may acquire specificity to other MHC-antigen (or neoepitope) complex depending on the sequences of the two distinct and separate peptides.

Most preferably, it is contemplated that the two peptides are selected such that, when peptides are grafted on and replaced portions of the endogenous T cell alpha chain and T cell beta chain of NKT cell, the endogenous T cell receptor of the NKT cell can be transformed to a T cell receptor specifically binds to cancer antigens or neoepitopes coupled with MHC molecules (e.g., MHC-I, MHC-II). Thus, in one embodiment, two peptides are at least a portion of an extracellular domain of T cell receptor alpha chain, and at least a portion of an extracellular domain of T cell receptor beta chain, respectively. Preferably, the portions of the extracellular domain of T cell receptor alpha and beta chains, together or separately, can bind to cancer antigens or neoepitopes (cancer-specific, patient-specific) presented on the antigen presenting cells (e.g., cancer cells) with an affinity of at least a K_{D} of at least equal or less than 10⁻⁶M, preferably at least equal or less than 10⁻⁷M, more preferably at least equal or less than 10⁻⁸M. In other embodiments, at least one of two peptides can include at least one or more single chain variable fragment (scFv), or a fragment of a whole antibody molecule, with an affinity of at least a K_{D} of at least equal or less than 10⁻⁶M, preferably at least equal or less than 10⁻⁷M, more preferably at least equal or less than 10⁻⁸M to cancer antigens or neoepitopes (cancer-specific, patient-specific). In these embodiments, the fragment of a whole antibody may include, but not limited to, Fab fragments, Fab′ fragments, F(ab′)2, disulfide linked Fvs (sdFvs), Fvs, and any fragment comprising either V_{H} segment and/or V_{L} segment. Where the antibody is an immunoglobulin, it is contemplated that the immunoglobulin can include any type (e.g., IgG, IgE, IgM, IgD, IgA and IgY) and any class (e.g., IgG1, IgG2, IgG3, IgG4, IgA1 and IgA2) of heavy chain or constant domain to constitute different types of immunoglobulin. In addition, the “antibody” can include, but not limited to a human antibody, a humanized antibody, a chimeric antibody, a monoclonal antibody, a polyclonal antibody.

Without wishing to be bound by any specific theory, the inventors contemplate that the genetically modified NKT cells expressing either the recombinant protein (CAR) or the protein complex (T cell receptor complex) that specifically recognize a cancer (neo)epitope increase the NKT cell immune response (e.g., cytotoxicity against the tumor cells), against the tumor by tumor-specific targeting. In addition, by removing CD1d-restriction from the NKT cells, the inventors contemplate that genetically modified NKT cells can be used further, as more NKT cells are recruited near the tumor, the NKT cells are expected to alter the microenvironment of the tumor via their immune surveillance function (e.g., by locally releasing cytokines, etc.).

NKT Cell Expressing a Fas Ligand and/or a CD40 Ligand:

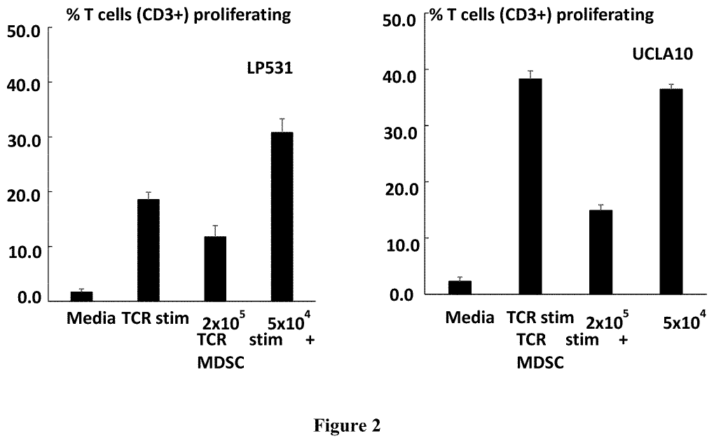

In many tumors, immune cell responses against the tumor cells are suppressed by a changed tumor microenvironment, which often includes accumulation of immunosuppressive regulatory T cells (Tregs) and myeloid-derived suppressor cells (MDSCs). As shown in FIG. 2, one of the immune suppression mechanism by MDSCs is by inhibiting T cell proliferation. Thus, in another inventive subject matter, the inventors contemplate that the NKT cells are genetically modified to induce cell death of Tregs or MDSCs to so change the tumor microenvironment to be less immune-suppressive. For example, in one preferred embodiment, NKT cells are genetically modified to express at least one of Fas ligand, a CD40 ligand, or both.

While any suitable forms of recombinant nucleic acid composition to encode Fas ligand and/or a CD40 ligand can be used, the inventors contemplate that, in some embodiments, the Fas ligand and a CD40 ligand can be encoded by a single nucleic acid comprising a plurality of segments, each of which encodes a distinct peptide. Thus, in one preferred embodiment, the nucleic acid composition includes a first nucleic acid segment encoding a Fas ligand (FasL, CD95L), and a second nucleic acid segment encoding a CD40 ligand (CD40L or CD154). Preferably, the first and second nucleic acid segments are mRNAs, which are separated by nucleic acid sequences encoding a type of 2A self-cleaving peptide (2A). As used herein, 2A self-cleaving peptide (2A) refers any peptide sequences that can provide a translational effect known as “stop-go” or “stop-carry” such that two sub-segments in the same mRNA fragments can be translated into two separate and distinct peptides. Any suitable types of 2A peptide sequences are contemplated, including porcine teschovirus-1 2A (P2A), thosea asigna virus 2A (T2A), equine rhinitis A virus 2A (E2A), foot and mouth disease virus 2A (F2A), cytoplasmic polyhedrosis virus (BmCPV 2A), and flacherie virus (BmIFV 2A).

Without binding to any specific theory, it is contemplated that the NKT cells expressing Fas ligand and/or a CD40 ligand triggers a Fas-dependent cell death of Tregs or MDSCs so that the accumulation of Tregs or MDSCs in the tumor microenvironment is prevented or the number of Tregs or MDSCs in the tumor microenvironment is reduced. In addition, the NKT cell expressing Fas ligand and/or a CD40 ligand can further facilitate the cell death of Tregs or MDSCs by releasing cell death-triggering cytokines (e.g., IL-2) near the Tregs or MDSCs. Thus, the inventors further contemplate, in a preferred embodiment, one or more cytokine (e.g., IL-2) facilitating cell death of Tregs or MDSCs can be concurrently administered to or expressed in the tumor microenvironment upon the administration of genetically modified NKT cells expressing Fas ligand and/or a CD40 ligand. It is expected that induction of Fas-dependent cell death of Tregs or MDSCs may further trigger changes of tumor microenvironment to less immune-suppressive such that the tumor cells can be more susceptible to subsequent immunotherapy or further treatment with naive or genetically modified NKT cells.

Introduction of Recombinant Nucleic Acid into NKT Cells

The recombinant nucleic acids (either encoding the recombinant protein, the protein complex, or FasL/CD40L as described) can be introduced into NKT cells by any suitable means. Preferably, the recombinant nucleic acid is introduced such that it can be present as a stable or transient extrachromosomal unit (e.g., as plasmid, yeast artificial chromosome, etc., which may have replicating capability) in the transfected cell. The suitable vector includes, but not limited to, any mammalian cell expression vector and a viral vector, depending on the methodology of introducing the recombinant nucleic acid to the cells. Alternatively, where the recombinant nucleic acid(s) is/are RNA, the nucleic acid may be transfected into the cells. It should also be recognized that the manner of recombinant expression is not limited to a particular technology so long as the modified cells are capable of producing the chimeric protein in a constitutive or inducible manner. Therefore, the cells may be transfected with linear DNA, circular DNA, linear RNA, a DNA or RNA virus harboring a sequence element encoding the chimeric protein, etc. Viewed form a different perspective, transfection may be performed via ballistic methods, virus-mediated methods, electroporation, laser poration, lipofection, genome editing, liposome or polymer-mediated transfection, fusion with vesicles carrying recombinant nucleic acid, etc.

Thus, it should also be appreciated that the recombinant nucleic acid may be integrated into the genome (via genome editing or retroviral transfection) or may be present as a stable or transient extrachromosomal unit (which may have replicating capability). For example, the recombinant nucleic acid that is used to transfect the cytotoxic cell may be configured as a viral nucleic acid and suitable viruses to transfect the cells include adenoviruses, lentiviruses, adeno-associated viruses, parvoviruses, togaviruses, poxviruses, herpes viruses, etc. Alternatively, the recombinant nucleic acid may also be configured as extrachromosomal unit (e.g., as plasmid, yeast artificial chromosome, etc.), or as a construct suitable for genome editing (e.g., suitable for CRiPR/Cas9, Talen, zinc-finger nuclease mediated integration), or may be configured for simple transfection (e.g., as RNA, DNA (synthetic or produced in vitro), PNA, etc.). Therefore, it should also be noted that the cells may be transfected in vitro or in vivo.

The authors contemplate that the genetically modified NKT cells expressing a recombinant chimeric antigenic receptor (CAR), a T cell receptor complex, or a hybrid T cell receptor complex, express those molecules in replacement of endogenous NKT cell T cell receptors such that NKT cells may lose its specificity (or restriction) to CD1d-(lipid) antigen complex and may acquire specificity to other MHC-antigen (or neoepitope) complex depending on the sequences of the two distinct and separate peptides. In such embodiments, the genes encoding alpha and beta chains of T cell receptor in NKT cells can be knocked out (e.g., deletion, etc.) and nucleic acids encoding recombinant chimeric antigenic receptor (CAR) or a T cell receptor complex can be introduced to the NKT cells (e.g., using a viral vector, etc.) such that the introduced foreign chimeric antigenic receptor (CAR) or a T cell receptor complex can be expressed in the NKT cells instead of knocked-out endogenous T cell receptors.

It is preferred that the nucleic acids encoding a CAR or a T cell receptor complex can be knocked-in at the targeted locus and replaces at least 50%, at least 60%, at least 70%, at least 80%, at least 90% of endogenous T cell receptor alpha or beta chain or portions thereof so that the expression of endogenous T cell receptor can be virtually abolished (e.g., less than 20%, less than 10%, less than 5%, less than 1%) and/or any functional or non-functional fragment(s) of endogenous T cell receptor that can be competitive with introduced proteins (CAR or a T cell receptor complex) may not be expressed.

Inventors further contemplate that any suitable numbers of recombinant nucleic acids and knock-in loci can be used to knock-in the recombinant nucleic acid(s). For example, where the genetically modified NKT cell expresses CAR, the CAR can be encoded by a single nucleic acid, which can be further inserted into one or more vectors to be integrated into one or more loci of the NKT cell genome. Thus, in this example, the recombinant nucleic acid encoding CAR can be integrated one of loci encoding T cell receptor alpha chain (chromosome 14) or T cell receptor beta chain (chromosome 7) or both. For other example, where the genetically modified NKT cell expresses recombinant T cell receptor complex, the recombinant T cell receptor complex can be encoded by a single nucleic acid (with a plurality nucleic acid segments as described above), which can be further inserted into one or more vectors to be integrated into one or more loci of the NKT cell genome. Thus, in this example, the recombinant nucleic acid encoding the recombinant T cell receptor complex can be integrated into one of loci encoding T cell receptor alpha chain (chromosome 14) or T cell receptor beta chain (chromosome 7) or both. For still other example, where the genetically modified NKT cell expresses recombinant T cell receptor complex, the recombinant T cell receptor complex can be encoded by a plurality of nucleic acids, each of which can be further inserted into one or more vectors to be integrated into one or more loci of the NKT cell genome. Thus, in this example, the recombinant nucleic acid encoding alpha chain of recombinant T cell receptor can be integrated into the locus encoding endogenous T cell receptor alpha chain, and the recombinant nucleic acid encoding beta chain of recombinant T cell receptor can be integrated into the locus encoding endogenous T cell receptor beta chain, respectively.

For still other example, where the genetically modified NKT cell expresses the hybrid T cell receptor, the two distinct peptide of the hybrid T cell receptor can be encoded by two distinct nucleic acid segments located in one or more vectors. Preferably, the recombinant nucleic acid encoding the first peptide is enclosed in the cassette targeting the locus encoding T cell receptor alpha chain (chromosome 14) and the recombinant nucleic acid encoding the second peptide is enclosed in the cassette targeting the locus encoding T cell receptor beta chain (chromosome 7), such that both endogenous alpha and beta chain can be replaced with the hybrid alpha and beta chains to acquire specificity to tumor antigens/neoantigen and lose (or decrease) the specificity (or restriction) to CD1d-presented lipid antigens.

The inventors further contemplate that the NKT cells to be genetically modified with the recombinant nucleic acids described above can be ex vivo expanded NKT cells, or specific types of NKT cells that are expanded ex vivo with specific glycolipid agonist(s) (e.g., α-GlcCer, β-ManCer, GD3, etc.) depending on the desired immune response against the tumor cells or tumor microenvironment. For example, NKT cells activated with β-ManCer can be genetically modified as described above to induce NKT-cell mediated immune response that is dependent on NOS (e.g., involving macrophage activity, etc.). For other example, NKT cells activated with α-GlcCer can be genetically modified as described above to induce NKT-cell mediated immune response that is independent of NOS (e.g., suppression of MDSC-mediated immune suppression, etc.). While above two types of cells can be used separately for distinct purpose of immune therapy, it is also contemplated that both types of NKT cells can be genetically modified with same or different nucleic acid construct to provide synergistic effect in the immune therapy.

### Expansion of NKT Cells Ex Vivo and Activation

Additionally, the population of isolated and enriched NKT cells can be further increased via ex vivo expansion of the NKT cells before and/or after the recombinant nucleic acid encoding CAR, recombinant T cell receptor complex, or peptides to form the hybrid T cell receptor is introduced. The ex vivo expansion of NKT cells can be performed in any suitable method with any suitable materials that can expand NKT cells at least 10 times, preferably at least 100 times in 7-21 days. For example, isolated and enriched NKT cells can be placed in a cell culture media (e.g., AIMV® medium, RPMI1640® etc.) that includes one or more activating conditions. The activating conditions may include addition of any molecules that can stimulate NKT growth, induce cell division of NKT, and/or stimulate cytokine release from NKT that can further expand NKT cells. It is contemplated that the activating conditions may vary depending on the timing of the ex vivo expansion and activation. For example, ex vivo expansion and activation of NKT cells can be performed using the activator of endogenous NKT T cell receptor or antibodies against the components of the endogenous NKT T cell receptor, before the endogenous NKT T cells are removed by knock-in of recombinant nucleic acid. However, after the endogenous NKT T cells are removed by knock-in of recombinant nucleic acid, it is contemplated that the activator of endogenous NKT T cell receptor or antibodies against the components of the endogenous NKT T cell receptor may not be used for effective ex vivo expansion and activation.

Thus, the activating molecules may include T cell receptor antibodies (e.g., anti-CD2, anti-CD3, anti-CD28, α-TCR-Vα24+ antibodies, preferably immobilized on beads, etc.), a glycolipid (e.g., α-GlcCer, β-ManCer, GD3, etc.), a glycolipid coupled with CD1 (e.g., CD1d, etc.) if the ex vivo expansion and activation is performed before the recombinant nucleic acid is introduced into the NKT cells. After the recombinant nucleic acid is introduced into the NKT cells, the activating molecules may include one or more cytokines (e.g., IL-2, IL-5, IL-7, IL-8, IL-12, IL-12, IL-15, IL-18, and IL-21, preferably human recombinant IL-2, IL-5, IL-7, IL-8, IL-12, IL-12, IL-15, IL-18, and IL-21, etc.) in any desirable concentration (e.g., at least 10 U/ml, at least 50 U/ml, at least 100 U/ml), etc. In some embodiments, the activation conditions may include culturing the isolated and enriched NKT cells with autologous or allogeneic peripheral blood mononuclear cells (PBMC) feeder cells.

In addition, the inventors also contemplate that the ex vivo expansion of the NKT cells can be directed to expansion of specific types of NKT cells by treating the NKT cells with different types of glycolipids (e.g., α-GlcCer, β-ManCer, GD3, etc.) that may trigger NKT cells having different profiles of cytokine release. For example, NKT cells can be treated with extracellular α-GlcCer ex vivo to induce expanded NKT cells to a particular type: IFN-γ producing NKT cells. For other example, NKT cells can be treated with extracellular β-ManCer ex vivo to induce expanded NKT cells to another particular type: NKT cells with TNF-α, iNOS-dependent antitumor activity.

With respect to the activating conditions, it is contemplated that the dose and schedule of providing activating conditions may vary depending on the initial number of NKT cells and the condition of NKT cells. In some embodiments, a single dose of cytokine (e.g., 100 U/ml) can be employed for at least 3 days, at least 5 days, at least 7 days, at least 14 days, at least 21 days. In other embodiments, the dose of cytokine may be increased or decreased during the expansion period (e.g., 200 U/ml for first 3 days and 100 U/ml for next 14 days, or 100 U/ml for first 3 days and 200 U/ml for next 14 days, etc.). Also it is contemplated that different types of cytokines can be used in combination or separately during the ex vivo expansion (e.g., IL-15 for first 3 days and IL-18 for next 3 days, or combination of IL-15 and IL-18 for 14 days, etc.).

Optionally, the expanded NKT cells can be further activated under conditions that will increase cytotoxicity. The condition to increases cytotoxicity include contacting the expanded NKT cells with T cell receptor antibodies (e.g., anti-CD2, anti-CD3, anti-CD28, α-TCR-Vα24+ antibodies, preferably immobilized on beads, etc.), a glycolipid (e.g., α-GlcCer, β-ManCer, GD3, etc.), or a glycolipid coupled with CD1 (e.g., CD1d, etc.), for a desired period (e.g., at least 1 hour, at least 6 hours, at least 24 hours, at least 3 days, at least 7 days, etc.). The cytotoxicity of the expanded and activated NKT cells can be determined by measuring the amount of cytokine release (e.g., IL-2, IL-13, IL-17, IL-21, TNF-α, etc.) from the NKT cells.

### Administration of NKT Cells to a Patient Having a Tumor.

The inventors also contemplate that ex vivo expanded and optionally activated NKT cells can be administered to a patient having a tumor (or suffering from autoimmune diseases or having a local infection with a microorganism, etc.). It is contemplated that the naive NKT cells (e.g., isolated, isolated and ex vivo expanded, genetically unmodified, etc.) and/or genetically engineered NKT cells can be formulated in any pharmaceutically acceptable carrier (e.g., as a sterile injectable composition) with a cell titer of at least 1×10³ cells/ml, preferably at least 1×10⁵ cells/ml, more preferably at least 1×10⁶ cells/ml, and at least 1 ml, preferably at least 5 ml, more preferably and at least 20 ml per dosage unit. However, alternative formulations are also deemed suitable for use herein, and all known routes and modes of administration are contemplated herein. As used herein, the term “administering” genetically naive NKT cells and/or genetically modified NKT cells refers to both direct and indirect administration of the naive NKT cells and/or genetically modified NKT cells formulation, wherein direct administration of naive NKT cells and/or genetically modified NKT cells is typically performed by a health care professional (e.g., physician, nurse, etc.), and wherein indirect administration includes a step of providing or making available the naive NKT cells and/or genetically modified NKT cell formulation to the health care professional for direct administration (e.g., via injection, etc.).

While the composition can comprise only naive NKT cells one type of genetically modified NKT cell (e.g., genetically modified NKT cells expressing CAR, genetically modified NKT cells expressing the recombinant T cell receptor complex, genetically modified NKT cells expressing the hybrid T cell receptor), it is also contemplated that the composition can comprise a mixture of naive NKT cells and genetically modified NKT cells, or a mixture of different types of genetically modified NKT cells. In this composition, the ratio of naive NKT cells and genetically modified NKT cells, or the ratio among the different types of genetically modified NKT cells may vary based on the type of cancer, age, gender, or health status of the patient, size of tumor, or NKT cell counts in the patient's blood. In some embodiments, the ratio of naive NKT cells and genetically modified NKT cells, or the ratio of two different genetically modified NKT cells is at least 1:1, at least 2:1, at least 3:1, at least 5:1, or at least 1:2, at least 1:3, or at least 1:5, and the ratio of three different genetically modified NKT cells can be at least 1:1:1, 1:2:1, 1:1:2, etc. Additionally, the naive NKT cells or genetically modified NKT cell may include or may be co-administered with a plurality of cytokine induced killer cells (CIK cells) to augment the cytotoxicity of the composition against the tumor cells.

In some embodiments, the naive NKT cells and/or genetically modified NKT cell formulation is administered via systemic injection including subcutaneous, subdermal injection, or intravenous injection. In other embodiments, where the systemic injection may not be efficient (e.g., for brain tumors, etc.), it is contemplated that the naive NKT cells and/or genetically modified NKT cell formulation is administered via intratumoral injection.

With respect to dose of the of naive NKT cells and/or genetically modified NKT cell formulation administration, it is contemplated that the dose may vary depending on the status of disease, symptoms, tumor type, size, location, patient's health status (e.g., including age, gender, etc.), and any other relevant conditions. While it may vary, the dose and schedule may be selected and regulated so that the naive NKT cells and/or genetically modified NKT cell does not provide any significant toxic effect to the host normal cells, yet sufficient to be effective to induce an cytotoxic effect and/or immune-modulatory effect against the tumor and/or the tumor microenvironment such that size of the tumor is decreased (e.g., at least 5%, at least 10%, at least 20%, etc.), the number of tumor cells is decreased, the phenotype of the tumor is changed (e.g., shape, change in gene expression, change in protein expression, change in post-translational modification of a protein, etc.), the accumulation of MDSC and/or Tregs is prevented (or stopped, decreased, etc.).

With respect to the schedule of administration, it is contemplated that it may also vary depending on the status of disease, symptoms, tumor type, size, location, patient's health status (e.g., including age, gender, etc.), and any other relevant conditions. In some embodiments, a single dose of naive NKT cells and/or genetically modified NKT cell formulation can be administered at least once a day or twice a day (half dose per administration) for at least a day, at least 3 days, at least a week, at least 2 weeks, at least a month, or any other desired schedule. In other embodiments, the dose of the naive NKT cells and/or genetically modified NKT cell formulation can be gradually increased during the schedule, or gradually decreased during the schedule. In still other embodiments, several series of administration of naive NKT cells and/or genetically modified NKT cell formulation can be separated by an interval (e.g., one administration each for 3 consecutive days and one administration each for another 3 consecutive days with an interval of 7 days, etc.).

In some embodiments, the administration of the naive NKT cells and/or genetically modified NKT cell formulation can be in two or more different stages: a priming administration and a boost administration. It is contemplated that the dose of the priming administration is higher than the following boost administrations (e.g., at least 20%, preferably at least 40%, more preferably at least 60%). Yet, it is also contemplated that the dose for priming administration is lower than the following boost administrations. Additionally, where there is a plurality of boost administration, each boost administration has different dose (e.g., increasing dose, decreasing dose, etc.).

In some embodiments, the dose and schedule of the naive NKT cells and/or genetically modified NKT cell formulation administration may be fine-tuned and informed by cellular changes of the infected cells or cancer cells. For example, after a cancer patient is administered with one or more dose of naive NKT cells and/or genetically modified NKT cell formulation, a small biopsy of the cancer tissue is obtained in order to assess any changes (e.g., upregulation of NKG2D ligand, apoptosis rate, etc.) resulted from interaction with naive NKT cells and/or genetically modified NKT cell formulation. The assessment of cellular changes can be performed by any suitable types of technology, including immunohistochemical methods (e.g., fluorescence labeling, in-situ hybridization, etc.), biochemical methods (e.g., quantification of proteins, identification of post-translational modification, etc.), or omics analysis. Based on the result of the assessment, the dose and/or schedule of the naive NKT cells and/or genetically modified NKT cell formulations can be modified (e.g., lower dose if excessive cytotoxicity is observed, etc.).

### Pretreatment to Tumor to Increase Effectiveness of NKT Cell Immune Response

It is contemplated that the genetically modified NKT cells are activated upon recognition of CD1d-antigen complex, tumor-associated antigens/neoepitope presented with MEW complex on the antigen presenting cells to elicit immune response against the antigen presenting cells by releasing multiple cytokines and chemokines (such as IL-2, Interleukin-13, Interleukin-17, Interleukin-21, and TNF-alpha). Thus, in addition to administering of naive NKT cells and/or genetically modified NKT cell to the patient, preferably to the tumor or tumor microenvironment, pre-conditioning of the tumor to promote a condition in which the tumor is more susceptible to administered NKT cells is especially contemplated.

For example, in some embodiments, the tumor cells can be preconditioned to NKT cell-susceptible (or responsive) conditions and followed by in vivo expansion of naive NKT cells. In such embodiments, the patient's naive NKT cells can be expanded by applying (preferably locally applying near the tumor) activating molecules, including but not limited to, cytokines (e.g., IL-2, IL-5, IL-7, IL-8, IL-12, IL-12, IL-15, IL-18, and IL-21, preferably human recombinant IL-2, IL-5, IL-7, IL-8, IL-12, IL-12, IL-15, IL-18, and IL-21, etc.) in any desirable concentration (e.g., at least 10 U/ml, at least 50 U/ml, at least 100 U/ml), T cell receptor antibodies (e.g., anti-CD2, anti-CD3, anti-CD28, α-TCR-Vα24+ antibodies, preferably immobilized on beads, etc.), a glycolipid (e.g., α-GlcCer, etc.), a glycolipid coupled with CD1 (e.g., CD1d, etc.). The amount of in vivo expanded NKT cells can be determined by counting the NKT cells from a biopsy tissue or from a locally collected bodily fluid.

While any suitable conditions that can increase the susceptibility or responsiveness of the cancer cells to the NKT cells are contemplated, it is most preferred that the tumor cells are treated with a condition to express the CD1d or CD1d coupled with a peptide or lipid antigen on the cell surface. In some embodiments, a recombinant nucleic acid encoding CD1d (wild-type or modified) can be introduced to the cancer cells so that the CD1d molecule is overexpressed in the tumor. Preferably, the recombinant nucleic acid encoding CD1d is inserted into a viral genome and introduced to the cancer cells. Any suitable virus to carry recombinant nucleic acid encoding CD1d is contemplated. The suitable virus include oncolytic virus, preferably genetically modified oncolytic virus presenting low immunogenicity to the host. For example, a preferred oncolytic virus includes genetically modified adenovirus serotype 5 (Ad5) with one or more deletions in its early 1 (E1), early 2b (E2b), or early 3 (E3) gene (e.g., E1 and E3 gene-deleted Ad5 (Ad5[E1]), E2b gene-deleted Ad5 (Ad5[E1,E2b], etc.). In one preferred virus strains having Ad5 [E1-, E2b-] vector platform, early 1 (E1), early 2b (E2b), and early 3 (E3) gene regions encoding viral proteins against which cell mediated immunity arises, are deleted to reduce immunogenicity. Also, in this strain, deletion of the Ad5 polymerase (pol) and preterminal protein (pTP) within the E2b region reduces Ad5 downstream gene expression which includes Ad5 late genes that encode highly immunogenic and potentially toxic proteins. Viewed from a different perspective and among other suitable viruses, particularly preferred oncolytic viruses include non-replicating or replication deficient adenoviruses.

Preferably, a recombinant nucleic acid encoding CD1d includes a nucleic acid segment encoding a signaling peptide that directs CD1d to the cell surface. Any suitable and/or known signaling peptides are contemplated (e.g., leucine rich motif, etc.). Preferably, the nucleic acid segment encoding CD1d is located in the upstream of the nucleic acid segment encoding signaling peptide such that the signal sequence can be located in C-terminus of the CD1d. However, it is also contemplated that the signaling peptide can be located in the N-terminus of CD1d, or in the middle of the CD protein.

Additionally, the recombinant nucleic acid encoding CD1d may include a nucleic acid segment encoding a peptide ligand of the CD1d (e.g., a hydrophobic short peptide), for example, p99. In this embodiment, the recombinant nucleic acid may include a first nucleic acid segment encoding CD1d and a second nucleic acid segment encoding p99, and the first and second nucleic acid segments are separated by a nucleic acid sequences encoding a type of 2A self-cleaving peptide (2A) so that the CD1d and p99 can be translated into two separate and distinct peptide, yet the expression of two peptides can be regulated under the same promoter. The inventors contemplate that the co-expressed CD1d and p99 are coupled intracellularly, trafficked together to the tumor cell surface, and trigger NKT cell activation when the NKT cell recognizes the tumor cells via CD1d-CD1d receptor interaction or MHC-epitope-T cell receptor (or CAR) interaction. It should be noted that in at least some instances, p99 may be bound to CD1d in an unorthodox manner that may disrupt conventional T cell recognition. However, as p99 appears to be a strong ligand with physiological signaling capability, other interactions (with T cells or other immune competent cells) are also contemplated herein.

Alternatively, the tumor cells pretreated with a recombinant nucleic acid encoding CD1d molecule (e.g., via oncolytic virus, etc.) can be further treated with one or more NKT cell agonists including, but not limited to, α-GalCer, β-mannosylceramide (β-ManCer), or GD3 (melanoma-derived ganglioside). In this embodiment, α-GalCer, β-ManCer, GD3, or any combination of those, can be locally applied (e.g., intratumorally injected, etc.) after the recombinant nucleic acid encoding CD1d is introduced to the tumor cells (e.g., at least 1 day after, at least 3 days after, at least 7 days after, etc.). The inventors contemplated that the surface-expressed CD1d binds to extracellular α-GalCer, β-ManCer, or GD3 to form a CD1d-α-GalCer complex, a CD1d-β-ManCer complex, or CD1d-GD3 complex and trigger NKT cell activation when the NKT cell recognizes the tumor cells via CD1d-CD1d receptor interaction or MHC-(neo)epitope-T cell receptor (or CAR) interaction. In an embodiment where multiple types of extracellular NKT cell agonists are treated to the tumor cells, it is contemplated that the extracellular NKT cell agonists can be treated sequentially (e.g., α-GalCer in day 1, β-ManCer in day 3, and GD3 in day 5, etc.). Yet, it is also contemplated that the multiple extracellular NKT cell agonists can be treated as a cocktail to the tumor cells for a single treatment or for multiple treatments.

In other embodiments, the tumor cells can be subjected to conditions or pretreated with any composition that can stress the tumor cells to increase expression of CD1d and/or CD1d coupled with a self-lipid molecule. For example, local heat shock treatment (e.g., at 42 degree celcius for 1 min, for 3 min, for 5 min, etc.), hypoxia, chemotherapy, exposure to toxins, and/or mechanical damage (e.g., partial surgical removal of cancer tissue, etc.) may be used to increase expression of CD1d and/or CD1d coupled with a self-lipid molecule.

In still other embodiments, the tumor cell can be pretreated with a drug that can facilitate CD1d surface expression on the tumor cell. For example, an inhibitor of HDAC that may trigger CD1d surface expression includes but not limited to hydroxamic acids (e.g., trichostatin A), cyclic tetrapeptides (e.g., trapoxin B, etc.), benzamides, electrophilic ketones, and the aliphatic acid (e.g. phenylbutyrate, valproic acid, etc.). In this embodiment, HDAC inhibitor can be locally (e.g., intratumoral injection, etc.) or systemically applied (e.g., orally administered, intraveneous injection, etc.) before the naive or genetically modified NKT cells are administered (e.g., at least 6 hours before, at least 24 hours before, at least 3 days before, etc.), or concurrently with the administration of the naive or genetically modified NKT cells.

In yet further aspects, the use of various compositions and compounds that include one or more naturally occurring or synthetic CD1d ligands (e.g., α-GalCer) is contemplated for use with various tumor targeting vehicles in vivo to activate endogenous or adoptively transferred NKT cells in the tumor vicinity as part of an immunization regimen. For example, and with respect to the naturally occurring or synthetic CD1d ligands, the same considerations as noted above apply. Such ligands can then be coupled to a tumor targeting moiety, and all known moieties that can specifically or preferentially target a tumor are deemed suitable for use here. Therefore, it should be recognized that the mechanism of targeting may be tumor epitope specific, or specific to one or more physiological characters of a tumor or tumor microenvironment.

For example, where the mechanism of targeting is tumor epitope specific, various scFv or antibody-based compositions may be employed, with or without an intermediary carrier. Among other items, scFv or antibodies may be chimeric polypeptides, or the CD1d ligand may be covalently bound to the scFv or antibody via a synthetic linker. In other examples, targeting may employ transcytosis in the tumor neovasculature and as such use albumin as a carrier of the CD1d ligand. Alternatively or additionally, the neovasculature may also be targeted via gp60-mediated transport and as such include at least an Fc portion. In still further aspects of such uses, the CD1d ligand may also be coupled to membranous carriers such as exosomes, liposomes, etc. (which may also include targeting entities such as scFv or antibodies).

Regardless of the particular components and manner of coupling, it is contemplated that such hybrid molecules will at least preferentially, and more typically selectively enrich in the tumor microenvironment and/or on a tumor cell due to the targeting portion. Upon co-location with the tumor cell or tumor mass, endogenous or adoptively transferred NKT cells will then be activated at the tumor and provide immunomodulatory function that reverses or at least reduces immune suppression (typically via interaction with MDSC). More relevant data and suggested models to apply above described subject matters are attached herein in Appendix A.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

What is claimed is:

1. A genetically engineered NKT cell, comprising a recombinant nucleic acid encoding a chimeric protein having 1) an extracellular single-chain variant fragment that specifically binds a tumor neoepitope, tumor associated antigen, or self-lipid, 2) an intracellular activation domain, and 3) a transmembrane linker coupling the extracellular single-chain variant fragment to the intracellular activation domain.

2. The genetically engineered NKT cell of claim 1, wherein the recombinant nucleic acid comprises:
a first nucleic acid segment encoding an extracellular single-chain variant fragment that specifically binds the tumor neoepitope, the tumor associated antigen, or the self-lipid;
a second nucleic acid segment encoding an intracellular activation domain;
a third nucleic acid segment encoding a linker between the extracellular single-chain variant fragment and the intracellular activation domain; and
wherein the first, second, and third segments are arranged such that the extracellular single-chain variant fragment, the intracellular activation domain, and the linker form a single chimeric polypeptide.

3. The genetically engineered NKT cell of claim 1, wherein the extracellular single-chain variant fragment comprises a V_{L} domain and a V_{H} domain of a monoclonal antibody against the tumor neoepitope, the tumor associated antigen, or the self-lipid.

4. The genetically engineered NKT cell of claim 3, wherein the extracellular single-chain variant further comprises a spacer between the V_{L} domain and the V_{H} domain.

5. The genetically engineered NKT cell of claim 1, further comprising a T cell receptor that specifically binds to CD1d.

6. The genetically engineered NKT cell of claim 1, wherein the NKT cell includes a Vα24-Jα18 T cell receptor.

7. The genetically engineered NKT cell of claim 1, wherein the intracellular activation domain comprises an immunoreceptor tyrosine-based activation motif (ITAM) that triggers ITAM-mediated signaling in the NKT cell.

8. The genetically engineered NKT cell of claim 1, wherein the intracellular activation domain comprises a portion of CD3ζ.

9. The genetically engineered NKT cell of claim 1, wherein the intracellular activation domain further comprises a portion of CD28 activation domain.

10. The genetically engineered NKT cell of claim 1, wherein the linker comprises a CD28 transmembrane domain or a CD3ζ transmembrane domain.

11. The genetically engineered NKT cell of claim 1, wherein the tumor epitope is patient-specific and tumor-specific.

12. The genetically engineered NKT cell of claim 1, wherein the recombinant nucleic acid replaces at least one of a portion of T cell receptor alpha locus and a portion of T cell receptor beta locus.

13. The genetically engineered NKT cell of claim 12, wherein the portion of T cell receptor alpha locus includes a nucleic acid sequence encoding a variable region of extracellular domain of T cell receptor alpha chain.

14. The genetically engineered NKT cell of claim 13, wherein the variable region of extracellular domain of T cell receptor alpha chain includes Vα24-Jα18 region of the T cell receptor alpha chain.

15. The genetically engineered NKT cell of claim 12, wherein the portion of T cell receptor beta locus includes a nucleic acid sequence encoding a variable region of extracellular domain of T cell receptor beta chain.

16. The genetically engineered NKT cell of claim 13, wherein the variable region of extracellular domain of T cell receptor alpha chain includes Vall region of the T cell receptor beta chain.

17. The genetically engineered NKT cell of claim 12, wherein the recombinant nucleic acid replaces the at least one of the portion of T cell receptor alpha locus and the portion of T cell receptor beta locus by a targeted genome editing nuclease.

18. The genetically engineered NKT cell of claim 17, wherein the targeted genome editing nucleases is Cas9 nuclease.

19. A genetically engineered NKT cell, comprising a recombinant nucleic acid encoding a protein complex having an α chain T cell receptor, a β chain T cell receptor, at least a portion of CD3δ, and at least a portion of CD3γ, wherein at least a portion of the α chain T cell receptor or a β chain T cell receptor is specific to a patient-specific, tumor-specific neoepitope, a tumor associated antigen, or a self-lipid.

20. The genetically engineered NKT cell of claim 19, wherein the recombinant nucleic acid comprises:
a first nucleic acid segment encoding an α chain T cell receptor and a β chain T cell receptor, the alpha and β chain receptor being separated by a first self-cleaving 2A peptide sequence;
a second nucleic acid segment encoding at least a portion of CD3δ and at least a portion of CD3γ, the at least portion of CD3δ and the at least portion of CD3γ being separated by a second self-cleaving 2A peptide sequence; and
wherein at least one of the α chain T cell receptor and the β chain T cell receptor together specifically bind a patient-specific, tumor-specific neoepitope or a tumor associated antigen, or CD1-lipid antigen complex.

21. The genetically engineered NKT cell of claim 19, wherein the first nucleic acid segment and the second nucleic acid segment are separated by a third self-cleaving 2A peptide sequence.

22. The genetically engineered NKT cell of claim 19, wherein the portion of CD3γ comprises an immunoreceptor tyrosine-based activation motif (ITAM).

23. The genetically engineered NKT cell of claim 19, wherein the portion of CD3δ comprises an immunoreceptor tyrosine-based activation motif (ITAM).

24. The genetically engineered NKT cell of claim 19, further comprising a T cell receptor that specifically binds to CD1d.

25. The genetically engineered NKT cell of claim 19, wherein the recombinant nucleic acid replaces at least one of a portion of T cell receptor alpha locus and a portion of T cell receptor beta locus

26. The genetically engineered NKT cell of claim 25, wherein the portion of T cell receptor alpha locus includes a nucleic acid sequence encoding a variable region of extracellular domain of T cell receptor alpha chain.

27. The genetically engineered NKT cell of claim 26, wherein the variable region of extracellular domain of T cell receptor alpha chain includes Vα24-Jα18 region of the T cell receptor alpha chain.

28. The genetically engineered NKT cell of claim 25, wherein the portion of T cell receptor beta locus includes a nucleic acid sequence encoding a variable region of extracellular domain of T cell receptor beta chain.

29. The genetically engineered NKT cell of claim 28, wherein the variable region of extracellular domain of T cell receptor alpha chain includes Vall region of the T cell receptor beta chain.

30. The genetically engineered NKT cell of claim 25, wherein the recombinant nucleic acid replaces the at least one of the portion of T cell receptor alpha locus and the portion of T cell receptor beta locus by a targeted genome editing nuclease.

31. The genetically engineered NKT cell of claim 30, wherein the targeted genome editing nucleases is Cas9 nuclease.

32. A pharmaceutical composition for treating a patient having a tumor, comprising:
a plurality of genetically engineered NKT cells according to claim 1 or claim 19.

33. A method of inducing an NKT cell immune response in a patient having a tumor, comprising:
obtaining from the patient a bodily fluid comprising a plurality of NKT cells;
enriching the NKT cells using a binding molecule specific to the plurality of NKT cells;
expanding a population of the NKT cells ex vivo; and
administering the expanded NKT cells to the patient in a dose and a schedule effective to induce an NKT cell immune response against the tumor.

34. The method of claim 33, wherein the bodily fluid is blood.

35. The method of claim 33, wherein the binding molecule is an antibody against Vα-24.

36. The method of claim 33, wherein the binding molecule is a portion of CD1d.

37. The method of claim 33, wherein the binding molecule is at least a portion of CD1d coupled with a lipid antigen.

38. The method of claim 33, wherein the binding molecule is at least a portion of CD1d coupled with a peptide antigen.

39. The method of claim 35, further comprising a step of further enriching the NKT cells using a portion of CD1d.

40. The method of claim 36, further comprising a step of further enriching the NKT cells using an antibody against Vα-24.

41. The method of claim 33, the expanding comprises treating the enriched NKT cells with a cytokine.

42. The method of claim 41, wherein the cytokine is selected from a group consisting of: IL-12, IL-15, IL-18, and IL-21.

43. The method of claim 33, further comprising providing a condition to the tumor to express a CD on a surface of the tumor.

44. The method of claim 43, wherein the condition comprises introducing a nucleic acid composition comprising a first nucleic acid segment encoding a CD1d.

45. The method of claim 44, wherein the nucleic acid composition further comprising a second nucleic acid segment encoding p99.

46. The method of claim 43, wherein the condition comprises a stress condition to the tumor.

47. The method of claim 43, wherein the condition comprises administering an inhibitor of HDAC to increase CD1d expression in the tumor.

48. The method of claim 43, wherein the NKT cells are genetically modified to express at least one of the following: a Fas ligand and a CD40 ligand.

49. The method of claim 33, wherein the NKT cell immune response against the tumor comprises reducing a size of the tumor.

50. The method of claim 33, wherein the NKT cell immune response against the tumor comprises suppressing activity of myeloid-derived suppressor cells.

51. The method of claim 33, wherein the administering the genetically modified NKT cell is performed by intravenous injection or intratumoral injection.

53. A method of suppressing an activity of myeloid-derived suppressor cells in a patient having a tumor, comprising;
administering a plurality of genetically modified NKT cells to the patient in a dose and a schedule effective to suppress the activity of myeloid-derived suppressor cells; and
wherein the genetically modified NKT cells express at least one of CD40L and Fas-L.

54. The method of claim 53, wherein the plurality of genetically modified NKT cells is CD1d-restricted T cells.

55. The method of claim 53, wherein the genetically modified NKT cells include a recombinant nucleic acid encoding at least one of CD40L and Fas-L.

56. The method of claim 53, further comprising providing a condition to the tumor to express a CD on a surface of the tumor.

57. The method of claim 56, wherein the condition comprises introducing a nucleic acid composition comprising a first nucleic acid segment encoding a CD1d.

58. The method of claim 57, wherein the nucleic acid composition further comprising a second nucleic acid segment encoding p99.

59. The method of claim 56, wherein the condition comprises a stress condition to the tumor.

60. The method of claim 56, wherein the condition comprises administering an inhibitor of HDAC to increase CD1d expression in the tumor.

61. The method of claim 53, wherein the administering the genetically modified NKT cell is performed by intravenous injection or intratumoral injection.

62. A method of inducing an NKT cell immune response in a patient having a tumor, comprising:
providing a genetically engineered NKT cell including a recombinant nucleic acid encoding chimeric protein having 1) an extracellular single-chain variant fragment that specifically binds a tumor neoepitope, a tumor associated antigen, or a self-lipid, 2) an intracellular activation domain, and 3) a transmembrane linker coupling the extracellular single-chain variant fragment to the intracellular activation domain; and
administering the genetically engineered NKT cells to the patient in a dose and a schedule effective to induce an NKT cell immune response against the tumor.

63. The method of claim 62, wherein the recombinant nucleic acid comprises:
a first nucleic acid segment encoding an extracellular single-chain variant fragment that specifically binds the tumor neoepitope, the tumor associated antigen, or the self-lipid;
a second nucleic acid segment encoding an intracellular activation domain;
a third nucleic acid segment encoding a linker between the extracellular single-chain variant fragment and the intracellular activation domain; and
wherein the first, second, and third segments are arranged such that the extracellular single-chain variant fragment, the intracellular activation domain, and the linker form a single chimeric polypeptide.

64. The method of claim 63, wherein the extracellular single-chain variant fragment comprises a V_{L} domain and a V_{H} domain of a monoclonal antibody against the tumor neoepitope, the tumor associated antigen, or the self-lipid.

65. The method of claim 64, wherein the extracellular single-chain variant further comprises a spacer between the V_{L} domain and the V_{H} domain.

66. The method of claim 62, the NKT cell further comprises a T cell receptor that specifically binds to CD1d.

67. The method of claim 62, wherein the NKT cell includes a Vα24-Jα18 T cell receptor.

68. The method of claim 62, wherein the intracellular activation domain comprises an immunoreceptor tyrosine-based activation motif (ITAM) that triggers ITAM-mediated signaling in the NKT cell.

69. The method of claim 62, wherein the intracellular activation domain comprises a portion of CD3ζ.

70. The method of claim 62, wherein the intracellular activation domain further comprises a portion of CD28 activation domain.

71. The method of claim 62, wherein the linker comprises a CD28 transmembrane domain or a CD3ζ transmembrane domain.

72. The method of claim 62, wherein the tumor epitope is patient-specific and tumor-specific.

73. The method of claim 62, wherein the NKT cell immune response against the tumor comprising reducing a size of the tumor.

74. The method of claim 62, wherein the NKT cell immune response against the tumor comprising suppressing activity of myeloid-derived suppressor cells.

75. The method of claim 74, the activity of myeloid-derived suppressor cells is suppressed by inducing a cell death of myeloid-derived suppressor cells.

76. The method of claim 62, wherein the recombinant nucleic acid further encodes at least one of CD40L and Fas-L.

77. The method of claim 62, wherein the NKT cells further include another recombinant nucleic acid encoding at least one of CD40L and Fas-L.

78. The method of claim 62, further comprising providing a condition to the tumor to express a CD on a surface of the tumor.

79. The method of claim 78, wherein the condition comprises introducing a nucleic acid composition comprising a first nucleic acid segment encoding a CD1d.

80. The method of claim 79, wherein the nucleic acid composition further comprising a second nucleic acid segment encoding p99.

81. The method of claim 78, wherein the condition comprises a stress condition to the tumor.

82. The method of claim 78, wherein the condition comprises administering an inhibitor of HDAC to increase CD1d expression in the tumor.

83. The method of claim 62, wherein the administering the genetically modified NKT cell is performed by intravenous injection or intratumoral injection.

84. The method of claim 62, further comprising obtaining a NKT cell from a bodily fluid of the patient.

85. The method of claim 84, wherein the NKT cells are obtained from the bodily fluid using an antibody against Vα-24.

86. The method of claim 84, wherein the NKT cells are obtained from the bodily fluid using a portion of CD1d.

87. The method of claim 84, wherein the NKT cells are obtained from the bodily fluid using a portion of CD1d coupled with a lipid antigen.

88. The method of claim 84, wherein the NKT cells are obtained from the bodily fluid using a portion of CD1d coupled with a peptide antigen.

89. The method of claim 62, further comprising enriching the NKT cells using a portion of CD1d or an antibody against Vα-24.

90. The method of claim 62, further comprising expanding a population of the genetically modified NKT cells ex vivo.

91. The method of claim 90, wherein the expanding comprises treating the enriched NKT cells with a cytokine.

92. The method of claim 91, wherein the cytokine is selected from a group consisting of: IL-12, IL-15, IL-18, and IL-21.

93. The method of claim 62, wherein the recombinant nucleic acid replaces at least one of a portion of T cell receptor alpha locus and a portion of T cell receptor beta locus.

94. The method of claim 93, wherein the portion of T cell receptor alpha locus includes a nucleic acid sequence encoding a variable region of extracellular domain of T cell receptor alpha chain.

95. The method of claim 94, the variable region of extracellular domain of T cell receptor alpha chain includes Vα24-Jα18 region of the T cell receptor alpha chain.

96. The method of claim 93, wherein the portion of T cell receptor beta locus includes a nucleic acid sequence encoding a variable region of extracellular domain of T cell receptor beta chain.

97. The method of claim 94, wherein the variable region of extracellular domain of T cell receptor alpha chain includes Vall region of the T cell receptor beta chain.

100. A method of inducing an NKT cell immune response in a patient having a tumor, comprising:
providing a genetically engineered NKT cell including a recombinant nucleic acid encoding recombinant nucleic acid encoding a protein complex having a chain T cell receptor, a β chain T cell receptor, at least a portion of CD3δ, and at least a portion of CD3γ; and
administering the genetically engineered NKT cells to the patient in a dose and a schedule effective to induce an NKT cell immune response against the tumor.

101. The method of claim 100, wherein the first nucleic acid segment and the second nucleic acid segment are separated by a third nucleic acid segment encoding a self-cleaving 2A peptide.

102. The method of claim 100, wherein the portion of CD3γ comprises an immunoreceptor tyrosine-based activation motif (ITAM).

103. The method of claim 100, wherein the portion of CD3δ comprises an immunoreceptor tyrosine-based activation motif (ITAM).

104. The method of claim 100, further comprising a T cell receptor that specifically binds to CD1d.

105. The method of claim 100, further comprising co-administering cytokine-induced killer cells with the genetically engineered NKT cells.

106. The method of claim 100, wherein the NKT cell immune response against the tumor comprising reducing a size of the tumor.

107. The method of claim 100, wherein the NKT cell immune response against the tumor comprising suppressing activity of myeloid-derived suppressor cells.

108. The method of claim 100, wherein the administering the genetically modified NKT cell is performed by intravenous injection or intratumoral injection.

109. The method of claim 100, wherein the recombinant nucleic acid encodes at least one of CD40L and Fas-L.

110. The method of claim 100, wherein the NKT cells further include another recombinant nucleic acid encoding at least one of CD40L and Fas-L.

111. The method of claim 100, further comprising providing a condition to the tumor to express a CD on a surface of the tumor.

112. The method of claim 111, wherein the condition comprises introducing a nucleic acid composition comprising a first nucleic acid segment encoding a CD1d.

113. The method of claim 112, wherein the nucleic acid composition further comprising a second nucleic acid segment encoding p99.

114. The method of claim 111, wherein the condition comprises a stress condition to the tumor.

115. The method of claim 111, wherein the condition comprises administering an inhibitor of HDAC to increase CD1d expression in the tumor.

116. The method of claim 100, wherein the recombinant nucleic acid replaces at least one of a portion of T cell receptor alpha locus and a portion of T cell receptor beta locus

117. The method of claim 116, wherein the portion of T cell receptor alpha locus includes a nucleic acid sequence encoding a variable region of extracellular domain of T cell receptor alpha chain.

118. The method of claim 117, wherein the variable region of extracellular domain of T cell receptor alpha chain includes Vα24-Jα18 region of the T cell receptor alpha chain.

119. The method of claim 116, wherein the portion of T cell receptor beta locus includes a nucleic acid sequence encoding a variable region of extracellular domain of T cell receptor beta chain.

120. The method of claim 117, wherein the variable region of extracellular domain of T cell receptor alpha chain includes Vall region of the T cell receptor beta chain.

121. The method of claim 117, wherein the recombinant nucleic acid replaces the at least one of the portion of T cell receptor alpha locus and the portion of T cell receptor beta locus by a targeted genome editing nuclease.

122. The method of claim 117, wherein the targeted genome editing nucleases is Cas9 nuclease.

123. A genetically engineered NKT cell, comprising a first recombinant nucleic acid sequence replacing a portion of T cell receptor alpha locus and encoding a first variable domain and a second recombinant nucleic acid sequence replacing a portion of T cell receptor beta locus and encoding a second variable domain, wherein the first and second domains collectively form a binding motif specific to a patient-specific, tumor-specific neoepitope or a tumor associated antigen.

124. The genetically engineered NKT cell of claim 123, wherein the portion of T cell receptor alpha locus includes a nucleic acid sequence encoding a variable region of extracellular domain of T cell receptor alpha chain.

125. The genetically engineered NKT cell of claim 124, wherein the variable region of extracellular domain of T cell receptor alpha chain includes Vα24-Jα18 region of the T cell receptor alpha chain.

126. The genetically engineered NKT cell of claim 123, wherein the portion of T cell receptor beta locus includes a nucleic acid sequence encoding a variable region of extracellular domain of T cell receptor beta chain.

127. The genetically engineered NKT cell of claim 126, wherein the variable region of extracellular domain of T cell receptor alpha chain includes Vall region of the T cell receptor beta chain.

128. The genetically engineered NKT cell of claim 123, wherein the recombinant nucleic acid replaces the at least one of the portion of T cell receptor alpha locus and the portion of T cell receptor beta locus by a targeted genome editing nuclease.

129. The genetically engineered NKT cell of claim 128, wherein the targeted genome editing nucleases is Cas9 nuclease.

130. Use of the genetically engineered NKT cells of any of claim 1-34, 35-58, or 224-234 for treating a tumor of a patient having the tumor.

131. Use of the pharmaceutical composition of claim 59 for treating a tumor of a patient having the tumor.

132. A pharmaceutical composition for treating a patient having a tumor, comprising:
a plurality of genetically engineered NKT cells according to any one of claims 224-234.

133. Use of the pharmaceutical composition of claim 237 for treating a tumor of a patient having the tumor.
